SEQUENCE LISTING (7 sequences)

SEQ ID NO: 1 - DNA, 4353 bp, Homo sapiens
  CDS (482)..(1564)
ttctctcacgaagccccgcccgcggagaggttccatattgggtaaaatctcggctctcggagagtcccgggagctgttctcgcgagagtactgcgggaggctcccgtttgctggctcttggaaccgcgaccactggagccttagcgggcgcagcagctggaacgggagtactgcgacgcagcccggagtcggccttgtaggggcgaaggtgcagggagatcgcggcgggcgcagtcttgagcgccggagcgcgtccctgcccttagcggggcttgccccagtcgcaggggcacatccagccgctgcggctgacagcagccgcgcgcgcgggagtctgcggggtcgcggcagccgcacctgcgcgggcgaccagcgcaaggtccccgcccggctgggcgggcagcaagggccggggagagggtgcgggtgcaggcgggggccccacagggccaccttcttgcccggcggctgccgctggaaaatgtctcaggagaggcccacgttctaccggcaggagctgaacaagacaatctgggaggtgcccgagcgttaccagaacctgtctccagtgggctctggcgcctatggctctgtgtgtgctgcttttgacacaaaaacggggttacgtgtggcagtgaagaagctctccagaccatttcagtccatcattcatgcgaaaagaacctacagagaactgcggttacttaaacatatgaaacatgaaaatgtgattggtctgttggacgtttttacacctgcaaggtctctggaggaattcaatgatgtgtatctggtgacccatctcatgggggcagatctgaacaacattgtgaaatgtcagaagcttacagatgaccatgttcagttccttatctaccaaattctccgaggtctaaagtatatacattcagctgacataattcacagggacctaaaacctagtaatctagctgtgaatgaagactgtgagctgaagattctggattttggactggctcggcacacagatgatgaaatgacaggctacgtggccactaggtggtacagggctcctgagatcatgctgaactggatgcattacaaccagacagttgatatttggtcagtgggatgcataatggccgagctgttgactggaagaacattgtttcctggtacagaccatattaaccagcttcagcagattatgcgtctgacaggaacaccccccgcttatctcattaacaggatgccaagccatgaggcaagaaactatattcagtctttgactcagatgccgaagatgaactttgcgaatgtatttattggtgccaatcccctggctgtcgacttgctggagaagatgcttgtattggactcagataagagaattacagcggcccaagcccttgcacatgcctactttgctcagtaccacgatcctgatgatgaaccagtggccgatccttatgatcagtcctttgaaagcagggacctccttatagatgagtggaaaagcctgacctatgatgaagtcatcagctttgtgccaccaccccttgaccaagaagagatggagtcctgagcacctggtttctgttctgttgatcccacttcactgtgaggggaaggccttttcacgggaactctccaaatattattcaagtgcctcttgttgcagagatttcctccatggtggaagggggtgtgcgtgcgtgtgcgtgcgtgttagtgtgtgtgcatgtgtgtgtctgtctttgtgggagggtaagacaatatgaacaaactatgatcacagtgactttacaggaggttgtggatgctccagggcagcctccaccttgctcttctttctgagagttggctcaggcagacaagagctgctgtccttttaggaatatgttcaatgcaaagtaaaaaaatatgaattgtccccaatcccggtcatgcttttgccactttggcttctcctgtgaccccaccttgacggtggggcgtagacttgacaacatcccacagtggcacggagagaaggcccataccttctggttgcttcagacctgacaccgtccctcagtgatacgtacagccaaaaaggaccaactggcttctgtgcactagcctgtgattaacttgcttagtatggttctcagatcttgacagtatatttgaaactgtaaatatgtttgtgccttaaaaggagagaagaaagtgtagatagttaaaagactgcagctgctgaagttctgagccgggcaagtcgagagggctgttggacagctgcttgtgggcccggagtaatcaggcagccttcataggcggtcatgtgtgcatgtgagcacatgcgtatatgtgcgtctctctttctccctcacccccaggtgttgccatttctctgcttacccttcacctttggtgcagaggtttcttgaatatctgccccagtagtcagaagcaggttcttgatgtcatgtacttcctgtgtactctttatttctagcagagtgaggatgtgttttgcacgtcttgctatttgagcatgcacagctgcttgtcctgctctcttcaggaggccctggtgtcaggcaggtttgccagtgaagacttcttgggtagtttagatcccatgtcacctcagctgatattatggcaagtgatatcacctctcttcagcccctagtgctattctgtgttgaacacaattgatacttcaggtgcttttgatgtgaaaatcatgaaaagaggaacaggtggatgtatagcatttttattcatgccatctgttttcaaccaactatttttgaggaattatcatgggaaaagaccagggcttttcccaggaatatcccaaacttcggaaacaagttattctcttcactcccaataactaatgctaagaaatgctgaaaatcaaagtaaaaaattaaagcccataaggccagaaactccttttgctgtctttctctaaatatgattactttaaaataaaaaagtaacaaggtgtcttttccactcctatggaaaagggtcttcttggcagcttaacattgacttcttggtttggggagaaataaattttgtttcagaattttgtatattgtaggaatcctttgagaatgtgattccttttgatggggagaaagggcaaattattttaatattttgtattttcaactttataaagataaaatatcctcaggggtggagaagtgtcgttttcataacttgctgaatttcaggcattttgttctacatgaggactcatatatttaagccttttgtgtaataagaaagtataaagtcacttccagtgttggctgtgtgacagaatcttgtatttgggccaaggtgtttccatttctcaatcagtgcagtgatacatgtactccagagggacagggtggaccccctgagtcaactggagcaagaaggaaggaggcagactgatggcgattccctctcacccgggactctccccctttcaaggaaagtgaacctttaaagtaaaggcctcatctcctttattgcagttcaaatcctcaccatccacagcaagatgaattttatcagccatgtttggttgtaaatgctcgtgtgatttcctacagaaatactgctctgaatattttgtaataaaggtctttgcacatgtgaccacatacgtgttaggaggctgcatgctctggaagcctggactctaagctggagctcttggaagagctcttcggtttctgagcataatgctcccatctcctgatttctctgaacagaaaacaaaagagagaatgagggaaattgctattttatttgtattcatgaacttggctgtaatcagttatgccgtataggatgtcagacaataccactggttaaaataaagcctatttttcaaatttagtgagtttctcaagtttattatatttttctcttgtttttatttaatgcacaatatggcattatatcaatatcctttaaactgtgacctggcatacttgtctgacagatcttaatactactcctaacatttagaaaatgttgataaagcttcttagttgtacattttttggtgaagagtatccaggtctttgctgtggatgggtaaagcaaagagcaaatgaacgaagtattaagcattggggcctgtcttatctacactcgagtgtaagagtggccgaaatgacagggctcagcagactgtggcctgagggccaaatctggcccaccacctgtttggtgtagcctgctaagaatggcttttacatttttaaatggttgggaaagaaaaaaaaagaagtagtagattttgtagcatgtgatgtaagtaatgtaaaacttaaattccagtatccataaataaagttttatgagaacaga

SEQ ID NO: 2 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 3 - DNA, 3560 bp, Mus musculus
  CDS (399)..(1481)
gatctggattcagagcggggcctccttggagctgttctcgcgagagttccgcgagaggctcccggccgctgcctgtgggatcgccgccactggagcccaagcggggcgctgaagcgcgagcgggtgtcttgcggcgtcggcgtgcgctccctccccggggagcggctgcaggaggaccgcggcgggagcagcctcgagccgtgcagccggctccggcaccttgccgacgctcgtaggagccgccgcggctgacaggggcggcgggtcgcagcctccacacctgcgcgggtggcgggcgcggggtccggtctgccgcgggcgggcgcagaggagagcgtgcggctgcaggcaggagcccccgctcggccacctcctcgccccgctgctgccgctggaagatgtcgcaggagaggcccacgttctaccggcaggagctgaacaagaccatctgggaggtgcccgaacgataccagaacctgtccccggtgggctcgggcgcctatggctcggtgtgtgctgcttttgatacaaagacggggcatcgtgtggcagttaagaagctgtcgagaccgtttcagtccatcattcacgccaaaaggacctaccgagagttgcgtctgctgaagcacatgaaacacgaaaatgtgattggtctgttggatgtgttcacacccgcaaggtcactggaggaattcaatgacgtgtacctggtgacccatctcatgggggcggacctgaacaacatcgtgaagtgccagaagctgaccgacgaccacgttcagtttctcatctaccagatcctccgagggctgaagtatatacattcggctgacataattcacagggacctaaagcccagcaacctagctgtgaacgaagactgtgagctcaagattctggattttgggctggctcggcacactgatgatgagatgacaggctacgtggctaccaggtggtaccgagccccagagatcatgctgaattggatgcactataaccagacagtggatatttggtccgtgggctgcatcatggctgagctgttgaccggaagaacgttgtttcctggtacagaccatattaaccagcttcagcagataatgcgtctgacggggacaccccctgcttatctcattaacaggatgccaagccatgaggcaagaaactacattcagtctctggcccagatgccgaagatgaacttcgcaaatgtatttattggtgccaatcccctggctgtcgacctactggagaagatgctcgttttggactcagataagaggatcacagcagcccaagctcttgcgcatgcctactttgctcagtaccacgaccctgatgatgagcctgttgctgacccttatgaccagtcctttgaaagcagggaccttctcatagatgagtggaagagcctgacctatgatgaagtcatcagctttgtgccaccaccccttgaccaagaagaaatggagtcctgagcacctggtttctgttctgtctatctcacttcactgtgaggggaagaccttctcatgggaactctccaaataccattcaagtgcctcttgttgaaagattccttcatggtggaagggggtgcatgtatgtgttagtgtttgtgtgtgtgtgtgtgtctgtctgttcgtctgtccacctatctttgtggaagtcactgtgatggtagtgactttatgagttgtgaatggtccttggcagtctgcctgctttctcagagtctgggcaggccgatgggaactgtcatctccttagggatgtgtgtgttcagtgcaaagtaagaaatatgaaaatatccctgttcttagttaccttgccactttggcttctcctgtggccctgcctttaccatatcagtgacagagagaggctgcttcaggtctgaggctatccctcagccatgcataaagtccaagagaaccaactggctcctggtctctagcctgtgaccggcttgcttaatgtcctcagaacctgacaggtatgttcaaaactgtcagtctgtttgtgccttaaaagggtgagaagggcgcgtagatagttacagagtctcagctgctgacgttctgagccaggcaagtgcacggggctgttggatggccagtggggagctggaaaaaacaaggcagcctttaggaaggccatggtgcatgtgtgtgcatgcgtgtatgtgcagccgccctccctcacttcaggagcaagctgtttgctgtgcttacccttcacctcagtgcagaggtctccagtgccgagcacaggcacctgccatcagtagttcctgtgtcatcttcacatctagcagagcacggatgtgtttgcatgctgtgctcttggagcttgtcctgtcttctggaagccctggacaaggcgtgtgaaggcttcccagaagttcctgtccacattgcctccgcccaccgacgccatgggcacactgctccctcctcctcctccagctactttgtgttgaacacaattgattctccaggtgctcatggtgcaggaaaacaggacagacagagagcacctgaacccttgccatctgatgtcaccaattcaggaaaacgagtcctctcctaggactatccccggttctggaaatcatgttctcctcactcatggtgacaagctaagaaagctgaacaaagggagagacgagagcgcctgaagccaggagctcctttactatctttctcaaaagggttgttagacacaaaccaagtcatcaaggccccgctcctctcctcggaagggtcccccaccccccggcagcttgacactgaatccagtgtcaatttggggagaaagcagttttgtcttggaattttgtatgttgtaggaatccttagagagtgtggttccttctgatggggagaaagggcaaattattttaatattttgtattttcacctttataaacatgaatcctcaggggtgaagaactgtttgcataattttctgaattttgagcactttgtgctatataaggacccatatttaagctttgtgtgcagtaagaaagtgtaaagccaattccagtgttggacgtgacaggtcttgtgtttaggtcaaggtgtctcctctcagtgcagggacatgcctgctctgtggggcaggcgaggaccctgaatcatttggagcccagaaggaggcagactggccaggtctcaccacctcagtgtgcagttcaactccatgccatcccatcaagatgggttagtagcagtgtctgtttttgaatgccaagtgtgatttccaacaattctgctctggttatttcattgaagacatctttgcacatgtgaccatgctgtgttaggggctgtgttccagggactggactcgaagctagaactggcagaagagttctggcatccacagcgcaatgctgccaccacccagtttcttcatcagaagacaagggaacgagaaaactgctgttcgtttgtatttgtgaacttggctgtaatctggtatgccataggatgtcagataataccactggttaaaataaagcctagttttcaaattcaaaaaaaaaaaaa

SEQ ID NO: 4 - protein, 360 aa, Mus musculus
MSQERPTFYRQELNKTIWEVPERYQNLSPVGSGAYGSVCAAFDTKTGHRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHINQLQQIMRLTGTPPAYLINRMPSHEARNYIQSLAQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 5 - RNA, 23 nt, Mus musculus
caaagugcuuacagugcagguag

SEQ ID NO: 6 - RNA, 23 nt, Mus musculus
caaagugcuaacagugcagguag

SEQ ID NO: 7 - RNA, 21 nt, Mus musculus
uaaagugcugacagugcagau

(19) United States
(12) Patent Application Publication
(10) Pub. No.: US 2015/0104870 A1
(43) Pub. Date: Apr. 16, 2015

(54) NEURONAL DIFFERENTIATION PROMOTER

(71) Applicant: KEIO UNIVERSITY, Tokyo (JP)

(72) Inventors: Hayato Kaneda, Yokohama-shi (JP); Hideyuki Okano, Tokyo (JP); Takuya Shimazaki, Tokyo (JP)

(21) Appl. No.: 14/397,376

(22) PCT Filed: Apr. 26, 2013
(86) PCT No.: PCT/JP2013/062490

§ 371 Date: Oct. 27, 2014

(30) Foreign Application Priority Data
Apr. 27, 2012 (JP) 2012-103875

(51) Int. Cl. C12N 5/0797; A61K 31/4439; C12N 5/0793; C12N 15/113

(52) U.S. Cl. CPC C12N 5/0623; C12N 15/1137; A61K 31/4439; C12N 5/0619; C12N 2310/141; C12N 2501/999; C12N 2501/65; C12N 2501/727; C12N 2506/08
USPC 435/377; 536/24.5; 546/274.1

(57) ABSTRACT

The present invention provides an agent for promoting neuronal differentiation of neural stem cells, which contains a p38 inhibitor. In addition, the present invention provides a method of promoting neuronal differentiation of neural stem cells, which includes cultivating neural stem cells in the presence of a p38 inhibitor. Neural stem cells are gliogenic or neurogenic. The p38 inhibitor is a nucleic acid which hybridizes to DNA or mRNA encoding p38 under physiological conditions, thereby inhibiting the transcription and/or translation thereof, an expression vector of the nucleic acid, a low molecular p38 inhibitor and the like.

### TECHNICAL FIELD

The present invention relates to an agent for promoting neuronal differentiation of neural stem cells, and a method of promoting neuronal differentiation of neural stem cells.

### BACKGROUND ART

Central nervous system (CNS) injury and disease have gained new prominence in modern medicine. Recent progress in stem cell biology, typified by induced pluripotent stem cell (iPSC) technology, has drawn attention to stem cells as innovative resources for transplantation therapy and individualized drug screening. Multipotent neural stem cells (NSCs), which give rise to all types of neural cells within the CNS, are now readily obtained from iPSCs. However, the specific and efficient induction of homogeneous target cell populations from NSCs remains difficult due to the complex mechanisms regulating NSC development and differentiation. Therefore, further identification of the means by which specific cell types are generated from NSCs is urgently required to facilitate their therapeutic application.

The present inventors have been using a recently-developed embryonic stem cell (ESC)-derived neurosphere culture system to investigate the molecular mechanisms governing NSC differentiation (non-patent document 1, patent document 1). Although NSCs possess the multipotency to differentiate into neurons and glial cells, neurogenesis largely precedes gliogenesis during CNS development in vertebrates. This neurogenesis-to-gliogenesis switch requires the temporal identity transition of NSCs (non-patent document 2). Importantly, the neurosphere culture system developed by the present inventors can recapitulate neural development in vivo, including this temporal regulation. Using this system, the present inventors previously discovered that Coup-tfI and II (Coup-tfs; also known as Nr2f1 and Nr2f2) act as critical molecular switches for the differentiation potential transition of NSCs (non-patent document 1, patent document 1).

### DOCUMENT LIST

### Patent Document

patent document 1: WO2010/018652

### Non-Patent Documents

non-patent document 1: Nat. Neurosci. 11, 1014-1023 (2008)
non-patent document 2: Nat. Neurosci. 9, 743-751 (2006)

### SUMMARY OF THE INVENTION

### Problems to be Solved by the Invention

An object of the present study is to elucidate the molecular mechanism involved in the competence change of NSCs and provide a technique for improving the differentiation efficiency of neural stem cells into neurons.

### Means of Solving the Problems

The present inventors have conducted intensive studies in an attempt to solve the aforementioned problems and identified that micro RNA-17 (miR-17)/106-p38 pathway is an effector of transition of neural stem cells from a neurogenic type to a gliogenic type. Overexpression of miR-17 inhibited acquisition of gliogenic competence, and restored the neuropotency of neural stem cells without accompanying changes in the epigenetic status of glial marker gene promoter, in NSCs in which developmental stage has progressed up to the period when NSCs are generally differentiated into gliogenic NSCs. They have further identified that the direct target of miR-17/106 is p38. Restoration of neuropotency by p38 inhibition was achieved in mouse and human NSCs. They have conducted further studies based on these findings and completed the present invention.

Accordingly, the present invention relates to the following.
[1] An agent for promoting neuronal differentiation of neural stem cells, comprising a p38 inhibitor.
[2] The agent of [1], wherein the neural stem cell is gliogenic.
[3] The agent of [1], wherein the neural stem cell is neurogenic.
[4] The agent of any of [1]-[3], wherein the p38 inhibitor is a nucleic acid which hybridizes, under physiological conditions, to a DNA or mRNA encoding p38, thereby inhibiting the transcription and/or translation thereof, an expression vector of the nucleic acid, or a low molecular p38 inhibitor.
[5] The agent of [4], wherein the nucleic acid is an siRNA, miRNA or a precursor thereof, or an antisense nucleic acid targeting p38.
[6] The agent of [5], wherein the nucleic acid is miR-17, miR-106a, miR-106b, or a precursor of said miRNAs.
[7] The agent of [4], wherein the p38 inhibitor is SB203580.
[8] A method of promoting neuronal differentiation of neural stem cells, comprising cultivating neural stem cells in the presence of a p38 inhibitor.
[9] The method of [8], wherein the neural stem cells are gliogenic.
[10] The method of [8], wherein the neural stem cells are neurogenic.
[11] The method of any of [8]-[10], wherein the p38 inhibitor is a nucleic acid which hybridizes, under physiological conditions, to a DNA or mRNA encoding p38, thereby inhibiting the transcription and/or translation thereof, an expression vector of the nucleic acid, or a low molecular p38 inhibitor.
[12] The method of [11], wherein the nucleic acid is an siRNA, miRNA or a precursor thereof, or an antisense nucleic acid targeting p38.
[13] The method of [12], wherein the nucleic acid is miR-17, miR-106a, miR-106b, or a precursor of said miRNAs.
[14] The method of [11], wherein the p38 inhibitor is SB203580.
[15] A method of converting a gliogenic neural stem cell to a neurogenic neural stem cell, comprising cultivating a gliogenic neural stem cell in the presence of a p38 inhibitor.
[16] A method of promoting neuronal differentiation of neural stem cells in a mammal, comprising administering an effective amount of a p38 inhibitor to said mammal.
[17] A prophylactic or therapeutic agent for a disorder, for which promotion of neuronal differentiation of neural stem cells is required, which comprises a p38 inhibitor.
[18] A method of preventing or treating a disorder, for which promotion of neuronal differentiation of neural stem cells is required, in a mammal, comprising administering an effective amount of a p38 inhibitor to the mammal.
[19] A p38 inhibitor for use in the prophylaxis or treatment of a disorder, for which promotion of neuronal differentiation of neural stem cells is required.
[20] Use of a p38 inhibitor for the production of a prophylactic or therapeutic agent for a disorder, for which promotion of neuronal differentiation of neural stem cells is required.

### Effect of the Invention

According to the present invention, differentiation of a neural stem cell into a neuron can be promoted by a new mechanism of p38 suppression. This results from the restoration of neurogenic competence in gliogenic neural stem cells, and neuronal differentiation can be induced exclusively.

### BRIEF DESCRIPTION OF THE DRAWINGS

FIG. 1 shows representative immunocytochemical images of differentiated p2 neurospheres derived from NSCs infected with lentiviruses expressing microRNAs and GFP at the primary (p0) neurosphere stage. MiR-LacZ microRNA was used as a control. Neurospheres were immunostained with antibodies against βIII-tubulin, GFAP and GFP. Scale bar, 50 μm. The graph shows the percentage of βIII-tubulin-positive (βIII-tubulin+) neurons among virus-infected cells differentiated from p2 neurospheres (t test, n (≧100 cells)=3, *P<0.05 and ***P<0.001). Bars and error bars represent the mean+s.e.m for each condition.

FIG. 2 shows the sequence comparison of microRNAs encoded by the miR-17-92, miR-106b-25 and miR-106a-363 clusters. The nucleotides identical to the corresponding nucleotides in miR-17 are shaded.

FIG. 3 shows immunocytochemical images of miR-17 overexpressing p2 neurospheres. MiR-17-overexpressing p2 neurospheres did not undergo gliogenesis in response to LIF (10 ng/ml) and BMP2 (100 ng/ml). The graph shows the percentage of βIII-tubulin-positive (βIII-tubulin+) neurons among virus-infected cells differentiated from p2 neurospheres (t test, n (≧100 cells)=3, ***P<0.001). Scale bar, 50 μm. Bars and error bars represent the mean+s.e.m for each condition.

FIG. 4 shows the analysis results of the CpG methylation status of the Gfap promoter by bisulfite sequencing. The methylation frequency of 10 CpG sequences on the Gfap promoter, including the CpG site in the STAT3-binding sequence as indicated by the arrows, was examined. The graphs show the percentages of methylated CpG cytosines in the whole Gfap promoter (left graph) and the STAT3-binding region (right graph) (t test, n (≧10 clones)=3, NS (not significant), P>0.05). Bars and error bars represent the mean+s.e.m for each condition.

FIG. 5 shows immunocytochemical images of the cells differentiated from Coup-tfs-knockdown, miR-17-suppressed p0 neurospheres. Overexpression of TuD-miR-17 (GFP-positive cells) cancelled the neuronal differentiation-promoting action in Coup-tfs knockdown p2 neurospheres and restored differentiation into astrocytes as assessed by reduction in βIIItubulin-positive nerve cells and increased GFP immunostaining. The graph shows the percentage of neurons (t test, n (≧100 cells)=3, **, *P<0.05). Scale bar, 50 μm. Bars and error bars represent the mean+s.e.m for each condition.

FIG. 6 shows immunocytochemical images of the cells differentiated from TuD-miR-17 overexpressing p0 neurospheres in the presence of glial differentiation inducing factors. Overexpression of TuD-miR-17 caused precocious gliogenesis from p0 neurospheres in the presence of LIF and BMP2. The graph shows the percentage of astrocytes (t test, n (≧100 cells)=3, *P<0.05). Scale bar, 50 μm. Bars and error bars represent the mean+s.e.m for each condition.

FIG. 7 shows the expression of miR-17 and miR-106b at E11.5 and P0 which was determined by in situ hybridization with specific locked nucleic acid (LNA) probes. Scale bar, 300 μm.

FIG. 8 shows immunocytochemical images of cerebral cortex sections of a mouse injected into the cerebral ventricle with lentiviruses for the overexpression of miR-LacZ or miR-17. The lentiviruses for the overexpression of miR-LacZ or miR-17 were injected in utero into the cerebral ventricle of mouse embryos at E10.5, and the fates of the infected cells were examined in the cerebral cortex at P30 (t test, n (≧250 cells, ≧three independent sections)=three independent embryos, **P<0.01 and ***P<0.001). Neuronal nuclei were stained with an antibody against NeuN. Arrowheads indicate non-neuronal cells, which include GFAP-positive astrocytes. Most GFAP-negative non-neuronal cells (‘Others’) appeared to be immature astrocytes. Higher magnification images of the cells indicated with arrows are shown as inserts. Scale bar, 50 μm.

FIG. 9 shows immunocytochemical images of the cells differentiated from SB203580-treated p2 neurospheres. Treatment with the p38 inhibitor SB203580 during their growth increased the percentage of neurons differentiated from p2 neurospheres (t test, n (≧150 cells)=3, *P<0.05). Scale bar, 50 μm. Bars and error bars represent the mean+s.e.m for each condition.

FIG. 10 shows immunocytochemical images of the cells differentiated from p2 neurospheres introduced with p38 specific shRNA. The p38-specific shRNA introduced by lentiviral infection increased the neuropotency of p2 neurospheres (t test, n (≧100 cells)=3, *P<0.05). Scale bar, 50 μm. Bars and error bars represent the mean+s.e.m for each condition.

FIG. 11 shows immunocytochemical images of the cells differentiated from p2 neurospheres overexpressing miR-17 and miR-17s-resistant p38 mRNA. Simultaneous overexpression of miR-17 and miR-17s-resistant p38 mRNA (yielding 3× HA-tagged N terminal p38 as the protein product) eliminated the effect of overexpressed miR-17 to promote neuronal differentiation in p2 neurospheres. Hence, most of the progenitor cells that exogenously express p38 were fated to become astrocytes once again (t test, n (≧150 cells)=3, *P<0.05). Arrows indicate GFP, HA-tagged p38 and GFAP triple-positive astrocytes. Arrowheads indicate GFP and βIII-tubulin double-positive neurons. Scale bar, 50 μm. Bars and error bars represent the mean+s.e.m for each condition.

FIG. 12 shows immunocytochemical images of the cells differentiated from p38 overexpressing p0 neurospheres in the presence of a glial differentiation inducing factors. Overexpression of HA-tagged p38 caused precocious gliogenesis from p0 neurospheres in the presence of LIF and BMP2 (t test, n (≧100 cells)=3, *P<0.05). Scale bar, 50 μm. Bars and error bars represent the mean+s.e.m for each condition. Bars and error bars represent the mean+s.e.m for each condition.

FIG. 13 shows immunocytochemical images of the cells differentiated from miR-17-overexpressing p3 neurospheres. Overexpression of miR-17 restored neuropotency of mouse p3 neurospheres (t test, n (≧100 cells)=3, ***P<0.001). Scale bar, 50 μm.

FIG. 14 shows the analysis results of the CpG methylation status of the Gfap promoter by bisulfite sequencing. The graphs show the percentages of methylated CpG cytosines in the whole Gfap promoter (left graph) and the STAT3-binding region (right graph) (t-test, n (≧10 clones)=3, NS, P>0.05).

FIG. 15 shows immunocytochemical images of the cells differentiated from human neurospheres treated with SB203580. Treatment of human neurospheres with the p38 inhibitor, SB203580, increased the percentage of neurons differentiated from the neurospheres (t-test, n (≧150 cells)=3, *P<0.05). Scale bar, 50 μm.

FIG. 16 shows immunocytochemical images of the cells differentiated from human neurosphere overexpressing p38, in the presence of a glial differentiation inducing factors. Overexpression of HA-tagged p38 in human neurospheres increased the percentage of astrocytes differentiated from the neurospheres in the presence of LIF and BMP2 (t test, n (≧100 cells)=3, *P<0.05). Scale bar, 50 μm. Bars and error bars represent the mean+s.e.m for each condition.

FIG. 17 is a graph showing that miR-17 binds to a binding site in the 3′ untranslated region (UTR) of mouse p38 mRNA. To confirm direct interaction between miR-17 and its binding site in 3′UTR of p38 mRNA, a reporter assay was performed. The miR-17 binding site in 3′UTR of p38 mRNA (to be referred to as “Mp38 sensor”; 2970-2992 of NM_{—}011951) was cloned into a mammal expression vector of the DsRed-Express2 reporter system. After transformation of HEK293 cells, lentiviruses expressing miR-LacZ or miR-17 were introduced into a stable transformant. One week later, the intracellular expression level of DsRed-Express2 was analyzed by FACS. Forced expression of miR-17 caused a decrease in DsRed-Express2 linked to the Mp38 sensor, as compared to miR-LacZ. (t-test, n (≧3000 cells)=3, **P<0.01). Bars and error bars represent the mean+s.e.m for each condition.

FIG. 18 shows the comparison results of p38 expression by Western blotting. The expression levels of p38 in p0, p1 and p2 neurospheres were examined by an anti-p38 antibody (Cell Signaling Technology 9212). miR-17 suppressed p38 expression at p2 stage (t-test, n=3, NS (not significant) p>0.05, *p<0.05 and **p<0.01; p1 *p=0.0281, p2 **p=0.0063, p2 miR-LacZ *p=0.0243, p2 miR-17 p=0.9704, *p=0.0393).

FIG. 19 shows the results indicating that p38 controls acquisition of gliogenic competence in the developing mouse forebrain. Overexpression of p38 resulted in the production of cell clusters having abnormal early expression of GFAP protein in VZ and SVZ (t-test, n (≧100 cells)=5 independent embryos, *p<0.05; *p=0.0284) (left Figure). Higher magnification images of the cells indicated with arrows are shown in the right Figure. In the left and right Figures, the photographs of the uppermost panel indicate GFP or HA, the photographs in the second panel from the top indicate GFAP, and the photographs in the third panel from the top show merged image of GFAP, and GFP or HA. The lowermost panel of the left Figure shows Hoechst staining. Scale bar: 50 μm. In the lower right graph, bars and error bars represent the mean+s.e.m.

FIG. 20 shows the results indicating that p38 controls acquisition of gliogenic competence in the developing mouse forebrain. The photograph of the upper panel of the Figure shows merged image of Acsbg1 and GFP, and the photograph of the lower panel shows Hoechst staining. The percentage of Acsbg1-expressing astrocytes decreased in VZ and SVZ due to shRNA-mediated knockdown of p38 (t-test, n (≧100 cells)=5 independent embryos, *p<0.05; *p=0.0158). Scale bar: 50 μm. Bars and error bars represent the mean+s.e.m in the graph.

### DESCRIPTION OF EMBODIMENTS

The present invention provides an agent for promoting neuronal differentiation of neural stem cells, which comprises a p38 inhibitor.

A p38 inhibitor means a substance that suppresses expression or function of p38.

p38, also called Mapk14 (mitogen-activated protein kinase 14), is a member of the MAP kinase family, and is a known protein kinase activated by stimulation by cytokine, UV irradiation, heat or osmotic pressure stress and the like.

In the present specification, p38 generally means one derived from a mammal. Examples of the mammal include, but are not limited to, rodents such as mouse, rat, hamster, guinea pig and the like, experiment animals such as rabbit and the like, domestic animals such as swine, bovine, goat, horse, sheep, mink and the like, pets such as dog, cat and the like, primates such as human, monkey, Macaca fascicularis, Macaca mulatta, marmoset, orangutan, chimpanzee and the like, and the like. p38 is preferably derived from primates (human etc.) or rodents (mouse etc.).

In the present specification, factors such as polypeptide, polynucleotide and the like, “derived from organism X” means that the polypeptide or polynucleotide has an amino acid sequence or polynucleotide sequence the same as or substantially the same as that of the polypeptide or polynucleotide naturally expressed in the organism X. Being “substantially the same” means that the interested amino acid sequence or nucleic acid sequence has 70% or more (preferably 80% or more, more preferably 90% or more, further preferably 95% or more, most preferably 99% or more) identity with the amino acid sequence or nucleic acid sequence of a factor naturally expressed in organism X, and maintains the activity of the protein.

The amino acid sequence and nucleotide sequence of p38 are known. The representative amino acid sequences of human p38 and mousep 38 and the polynucleotide sequences (cDNA or mRNA sequence) encoding same are as described below. In the parentheses, the accession numbers of GENBANK provided by NCBI are shown.

[Human p38]
amino acid sequence: accession number NP_{—}001306 (version NP_{—}001306.1) (SEQ ID NO: 2)
nucleotide sequence (cDNA sequence): accession number NM_{—}001315 (version NM_{—}001315.2) (SEQ ID NO: 1)
   [Mouse p38]
amino acid sequence: accession number NP_{—}001161980 (version NP_{—}001161980.1) (SEQ ID NO: 4)
nucleotide sequence (cDNA sequence): accession number NM_{—}001168508 (version NM_{—}001168508.1) (SEQ ID NO: 3)

In the present specification, nucleotide sequences are described as DNA sequences unless otherwise specified; however, when the polynucleotide is an RNA, thymine (T) should read as uracil (U) as appropriate.

Examples of the function of p38 include phosphorylation of serine or treonine in a substrate protein such as ATF2, MEF2C, MAX, CDC25B, p53 and the like.

Examples of the p38 inhibitor include a nucleic acid which hybridizes, under physiological conditions, to a DNA or mRNA encoding p38, thereby inhibiting the transcription and/or translation thereof, an expression vector of the nucleic acid, or a low-molecular-weight compound and the like.

Examples of the nucleic acid which hybridizes, under physiological conditions, to a DNA or mRNA encoding p38, thereby inhibiting the transcription and/or translation thereof include siRNA, micro RNA (miRNA) or a precursor thereof, an antisense nucleic acid and the like targeting p38.

Examples of the siRNA targeting p38 include
(A) an RNA comprising a nucleotide sequence complementary to the nucleotide sequence of an mRNA (mature mRNA or primary transcript) that encodes p38 or a partial sequence thereof having a length of 18 bases or more; and
(B) an RNA which comprises a nucleotide sequence having a length of 18 bases or more and capable of specifically hybridizing to an mRNA (mature mRNA or primary transcript) that encodes p38 in a cell of the target animal (preferably human or mouse), and suppresses transcription and/or translation of p38 by the hybridization.

In the present specification, “specific hybridization” means that a nucleic acid more strongly hybridizes to the target nucleotide than other nucleotides.

Examples of the nucleotide sequence of mRNA encoding p38 include the nucleotide sequence shown by SEQ ID NO: 1 (human p38), and the nucleotide sequence shown by SEQ ID NO: 3 (mouse p38).

What is called RNA interference (RNAi), a phenomenon in which transferring siRNA of a short double-stranded RNA and the like into a cell results in the degradation of mRNAs that are complementary to the RNA, has long been known to occur in nematodes, insects, plants and the like; recently, this phenomenon was confirmed as occurring also in animal cells [Nature, 411(6836): 494-498 (2001)], and this is attracting attention as a ribozyme-substitute technique.

A representative siRNA is a double-stranded oligo-RNA consisting of an RNA having a sequence complementary to the nucleotide sequence of the mRNA of the target gene or a partial sequence thereof (hereinafter, target nucleotide sequence) and a complementary strand for the RNA. A single-stranded RNA wherein a sequence complementary to the target nucleotide sequence (first sequence) and a complementary sequence for the same (second sequence) are linked via a hairpin loop portion, and wherein the first sequence forms a double-stranded structure with the second sequence by assuming a hairpin loop structure (small hairpin RNA: shRNA), also represents a preferred embodiment of siRNA.

The length of the portion complementary to the target nucleotide sequence, contained in the siRNA, is normally about 18 bases or more, preferably 19 bases or more, more preferably about 21 bases or more, and is not particularly limited as long as the expression of the target gene can be specifically suppressed. When the siRNA is longer than 23 bases, the siRNA undergoes degradation in cells to produce an siRNA of about 20 bases long; therefore, theoretically, the upper limit of the portion complementary to the target nucleotide sequence is the full length of the nucleotide sequence of an mRNA (mature mRNA or primary transcript) of the target gene. However, in view of the avoidance of interferon induction, the ease of synthesis, antigenicity problems and the like, the length of the complementary portion is, for example, about 50 bases or less, preferably about 25 bases or less, most preferably about 23 bases or less. Hence, the length of the complementary portion is, for example, normally, about 18-50 bases, preferably about 19 to about 25 bases, more preferably about 21 to about 23 bases.

In addition, the length of each RNA chain constituting siRNA is generally about 18 bases or more, preferably 19 bases or more, more preferably about 21 bases or more, and is not particularly limited as long as the expression of the target gene can be specifically suppressed. Theoretically, the length of each RNA chain does not have an upper limit. However, in view of the avoidance of interferon induction, the ease of synthesis, antigenicity problems and the like, the length of the siRNA is, for example, about 50 bases or less, preferably about 25 bases or less, most preferably about 23 bases or less. Hence, the length of each siRNA is, for example, about 18 to 50 bases, preferably about 19 to about 25 bases, more preferably about 21 to about 23 bases. The length of the shRNA is expressed as the length of the double-stranded moiety when the shRNA provides a double-stranded structure.

It is preferable that the target nucleotide sequence and the sequence complementary thereto contained in the siRNA be completely complementary to each other. However, in the presence of a base mutation at a position apart from the center of the siRNA (the mutation can be fall in the range of identity of at least 90% or more, preferably 95% or more), the cleavage activity by RNA interference is not completely lost, but a partial activity can remain. On the other hand, a base mutation in the center of the siRNA has a major influence to the extent that can extremely reduce the mRNA cleavage activity by RNA interference.

The siRNA may have an additional base that does not form a base pair at the 5′- and/or 3′-end. The length of the additional base is not particularly limited as long as siRNA can specifically suppress the expression of the target gene, and is normally 5 bases or less, for example, 2 to 4 bases. Although the additional base may be a DNA or an RNA, use of a DNA makes it possible to improve the stability of the siRNA. Examples of the sequences of such additional bases include, but are not limited to, the sequences ug-3′, uu-3′, tg-3′, tt-3′, ggg-3′, guuu-3′, gttt-3′, ttttt-3′, uuuuu-3′ and the like.

The length of the loop portion of the hairpin loop of the shRNA is not particularly limited, as long as the expression of the target gene can be specifically suppressed, and the length is normally about 5 to 25 bases. The nucleotide sequence of the loop portion is not particularly limited as long as a loop can be formed, and shRNA can specifically suppress the expression of the target gene.

Examples of the miRNA targeting p38 include miRNA containing, as a seed sequence, a nucleotide sequence complementary to a partial sequence (7 bases length) of the nucleotide sequence of mRNA encoding p38 (mature mRNA or primary transcript), and inhibiting transcription and/or translation of p38 can be mentioned.

miRNA representatively means what is called mature miRNA. The mature miRNA is an endogenous non-coding RNA having 20-25 bases, which is encoded on the genome. It is known that miRNA is transcribed as a primary transcript having a length of about several hundred-several thousand bases (Primary miRNA, hereinafter to be referred to as “Pri-miRNA”) from the miRNA gene on the genomic DNA, then processed to be a pre-miRNA (precursor miRNA) having a hairpin structure of about 60-70 bases, after which moves from the nucleus into the cytoplasm, further processed to be a double stranded mature miRNA of about 20-25 bases, a single strand of the double stranded mature miRNA forms a complex with a protein called RISC, and acts on the mRNA of the target gene to inhibit translation of the target gene.

In the present specification, the term “miRNA” is used as a concept encompassing not only endogenous miRNA but also a synthetic nucleic acid consisting of the same nucleotide sequence as the endogenous miRNA.

The seed sequence means the nucleotide sequence of the 5′-terminal 2nd-8th residues of the mature miRNA, and the seed sequence is known to determine the mRNA to be the target of miRNA.

miRNA targeting p38 can be obtained by searching, based on the nucleotide sequence of mRNA encoding p38 (for example, nucleotide sequence shown by SEQ ID NO: 1 or 3), for a miRNA having a seed sequence completely complementary to a partial sequence of the mRNA sequence (preferably, partial sequence of nucleotide sequence of 3′UTR of said mRNA), introducing the searched miRNA into the cell of p38 expressing cells, and selecting miRNA that suppressed expression of p38. The search of miRNA based on such target sequence can be performed based on target prediction programs such as TargetScan, PicTar, miRanda and the like.

Specific examples of miRNA targeting p38 include, but are not limited to, miR-17, miR-106a, miR-106b and the like. miR-17, miR-106a and miR-106b also include isomers of these miRNAs. The representative nucleotide sequences of miR-17, miR-106a and miR-106b are shown by SEQ ID NOs: 5, 6 and 7, respectively.

Also, a nucleic acid containing a nucleotide sequence having 85% or more, preferably 90% or more, more preferably 95% or more, identity with the nucleotide sequence shown by SEQ ID NO: 5, 6 or 7, and hybridizing, under physiological conditions, to an mRNA encoding p38, thereby inhibiting the transcription and/or translation thereof, is also encompassed in miRNA targeting p38.

The “identity” means a ratio (%) of identical nucleotide residues to all overlapping nucleotide residues in the optimal alignment where two nucleotide sequences are aligned using a mathematical algorithm known in the technical field (preferably, the algorithm considers introduction of gaps on one or both of the sequences for the best alignment).

In the present specification, nucleotide sequence identity can be calculated by, for example, aligning the two nucleotide sequences using the homology calculation algorithm NCBI BLAST-2 (National Center for Biotechnology Information Basic Local Alignment Search Tool) under the following conditions (gap open=5 penalties; gap extension=2 penalties; x_dropoff=50; expectation value=10; filtering=ON).

The nucleotide sequence having 85% or more identity with the nucleotide sequence shown by SEQ ID NO: 5, 6 or 7 is a nucleotide sequence which is the nucleotide sequence shown by SEQ ID NO: 5, 6 or 7 wherein 1 to 3 nucleotides are deleted, substituted, inserted or added, for example, (1) a nucleotide sequence which is the nucleotide sequence shown by SEQ ID NO: 5, 6 or 7 wherein 1-3 (preferably 1 or 2) nucleotides are deleted, (2) a nucleotide sequence which is the nucleotide sequence shown by SEQ ID NO: 5, 6 or 7 wherein 1-3 (preferably 1 or 2) nucleotides are added, (3) a nucleotide sequence which is the nucleotide sequence shown by SEQ ID NO: 5, 6 or 7 wherein 1-3 (preferably 1 or 2) nucleotides are inserted, (4) a nucleotide sequence which is the nucleotide sequence shown by SEQ ID NO: 5, 6 or 7 wherein 1-3 (preferably 1 or 2) nucleotides are substituted by other nucleotides, or (5) a nucleotide sequence wherein the mutations of the above-mentioned (1)-(4) are combined (in this case, total of mutated nucleotides is 1-3, preferably 1 or 2).

In the nucleotide sequence having 85% or more identity with the nucleotide sequence shown by SEQ ID NO: 5, 6 or 7, a seed sequence common to miR-17, miR-106a and miR-106b is preferably maintained.

The precursor of miRNA means a nucleic acid that can produce mature miRNA in a cell as a result of intracellular processing or cleavage of double stranded nucleic acid. As the precursor, pri-miRNA, pre-miRNA and the like can be mentioned. The pri-miRNA is a primary transcript (single strand RNA) of miRNA gene, and generally has a length of about several hundred-several thousand bases. pre-miRNA is a single strand RNA having a hairpin structure, which results from intracellular processing of pri-miRNA, and generally has a length of 90-110 base. As pri-miRNA or pre-miRNA of miRNA, for example, the nucleotide sequence thereof is disclosed in miRBase database: http://micro RNA.sanger.ac.uk/ produced by Sanger Institute and the like.

The siRNA, miRNA and a precursor thereof used in the present invention encompasses a nucleic acid at least partly containing RNA, which suppresses transcription and/or translation of p38 by RNA interference. Such nucleic acid is RNA, chimera nucleic acid of RNA and DNA (hereinafter to be referred to as chimera nucleic acid) or hybrid nucleic acid. As used herein, chimera nucleic acid refers to single strand or double strand nucleic acid containing RNA and DNA in a single nucleic acid, hybrid nucleic acid refers to double strand nucleic acid wherein one of the chains is RNA or chimera nucleic acid and the other chain is DNA or chimera nucleic acid.

The siRNA, miRNA and a precursor thereof used in the present invention are single strand or double strand. The embodiments of double strand include double strand RNA, double strand chimera nucleic acid, RNA/DNA hybrid, RNA/chimera nucleic acid hybrid, chimera nucleic acid/chimera nucleic acid hybrid and chimera nucleic acid/DNA hybrid. The nucleic acid of the present invention is preferably single strand RNA, single strand chimera nucleic acid, double strand RNA, double strand chimera nucleic acid, RNA/DNA hybrid, RNA/chimera nucleic acid hybrid, chimera nucleic acid/chimera nucleic acid hybrid or chimera nucleic acid/DNA hybrid, more preferably single strand RNA, single strand chimera nucleic acid, double strand RNA, double strand chimera nucleic acid, RNA/DNA hybrid, chimera nucleic acid/chimera nucleic acid hybrid, RNA/chimera nucleic acid hybrid or an artificial nucleic acid constituted to include a molecule equivalent to natural nucleic acid and the like.

The “antisense nucleic acid” refers to a nucleic acid comprising a nucleotide sequence capable of specifically-hybridizing with a target mRNA (mature mRNA or primary transcript) under physiological conditions for the cells that express the target mRNA, and capable of inhibiting the translation of the polypeptide encoded by the target mRNA in the hybridized state. The antisense nucleic acid may be a DNA or an RNA, and may be a DNA/RNA chimera, preferable DNA.

Examples of the antisense nucleic acid capable of specifically suppressing the expression of p38 include
(A) a nucleic acid comprising a nucleotide sequence complementary to the nucleotide sequence of an mRNA (mature mRNA or primary transcript product) that encodes p38 or a partial sequence thereof having a length of 12 bases or more; and
(B) a nucleic acid comprising a nucleotide sequence having a length of 12 bases or more which is capable of specifically hybridizing to an mRNA (mature mRNA or primary transcript) encoding p38, in the cell of an animal (preferably human) to be the subject of treatment, and capable of inhibiting, in the hybridized state, translation into p38 polypeptide, and the like.

The length of the portion that hybridizes with the target mRNA in the antisense nucleic acid is not particularly limited as long as the expression of p38 is specifically suppressed. Generally, the length is about 12 bases or more, and as long as the full-length sequence of the mRNA (mature mRNA or primary transcript) for the longest. In view of hybridization specificity, the length is, for example, preferably about 15 bases or more, more preferably about 18 bases or more. In view of the ease of synthesis, antigenicity problems and the like, the length of the portion that hybridizes with the target mRNA is normally about 200 bases or less, preferably about 50 bases or less, more preferably about 30 bases or less. Hence, the length of the portion that hybridizes with the target mRNA is, for example, about 12 to about 200 bases, preferably about 15 to about 50 bases, more preferably about 18 to about 30 bases.

The target nucleotide sequence for the antisense nucleic acid is not particularly limited, as long as the expression of p38 can be specifically suppressed. The sequence may be the full-length sequence of an mRNA (mature mRNA or primary transcript) of p38 or a partial sequence thereof (e.g., about 12 bases or more, preferably about 15 bases or more, more preferably about 18 bases or more), or an intron portion of the primary transcript.

The nucleotide sequence of the portion that hybridizes with the target mRNA in the antisense nucleic acid varies depending on the base composition of the target sequence, and has an identity of normally about 90% or more (preferably 95% or more, most preferably 100%) to the complementary sequence for the target sequence, so as to be capable of hybridizing with the p38 mRNA under physiological conditions.

The size of the antisense nucleic acid is normally about 12 bases or more, preferably about 15 bases or more, more preferably about 18 bases or more. In view of the ease of synthesis, antigenicity problems and the like, the size is normally about 200 bases or less, preferably about 50 bases or less, more preferably about 30 bases or less.

Furthermore, the antisense nucleic acid may be one not only capable of hybridizing with the p38 mRNA or primary transcript thereof to inhibit the translation, but also capable of binding to the p38 gene, which is a double-stranded DNA, to form a triple strand (triplex) and inhibit the transcription into p38.

In natural nucleic acids, the phosphodiester bond is easily cleaved by a nuclease present in the cells. Therefore, the siRNA, miRNA or a precursor thereof and an antisense nucleic acid used in the present invention may be modified to be a modified form resistant to the aforementioned nuclease. Examples of the modification to give a modified form include, but are not limited to, modified sugar chain moiety (e.g., 2′-O-methylation), modified base moiety, and modified phosphate or hydroxyl moieties (e.g., biotin, amino group, lower alkylamine group, acetyl group etc.).

siRNA and antisense nucleic acid targeting p38 can be prepared by determining the target sequence based on the mRNA sequence (e.g., the nucleotide sequence shown by SEQ ID NO:1 or 3) or chromosomal DNA sequence of p38, and synthesizing a nucleotide sequence complementary thereto using a commercially available automated DNA/RNA synthesizer (Applied Biosystems, Beckman and the like). An siRNA can be prepared by separately synthesizing a sense strand and antisense strand using an automated DNA/RNA synthesizer, denaturing the strands in an appropriate annealing buffer solution at about 90 to about 95° C. for about 1 minute, and then annealing at about 30 to about 70° C. for about 1 to about 8 hours. A longer double-stranded polynucleotide can be prepared by synthesizing complementary oligonucleotide strands in a way such that they overlap with each other, annealing the strands, and then performing ligation with a ligase.

The micro RNA targeting p38 can be obtained by searching, based on the mRNA sequence of p38 (for example, nucleotide sequence shown by SEQ ID NO: 1 or 3), for micro RNAs having a seed sequence complementary to a partial sequence of the mRNA sequence, preparing the searched micro RNAs by an automated DNA/RNA synthesizer (Applied Biosystems, Beckman and the like), introducing same into p38-expressing cells, and selecting a micro RNA actually suppressing the expression of p38.

The agent for promoting neuronal differentiation of the present invention can also contain, as an active ingredient, an expression vector capable of expressing (encoding) a nucleic acid which hybridizes, under physiological conditions, to a DNA or mRNA encoding p38, thereby inhibiting the transcription and/or translation thereof. In the expression vector, the nucleic acid which hybridizes, under physiological conditions, to a DNA or mRNA encoding p38, thereby inhibiting the transcription and/translation thereof, or a nucleic acid (preferably DNA) encoding same is operably linked to a promoter capable of exhibiting promoter activity in the cells (e.g., neural stem cell) of an mammal (preferably human or mouse) of application subject.

The promoter used is not particularly limited as long as it is functional in cells of the mammal of application subject. As the promoter, pol I promoter, pol II promoter, pol III promoter and the like can be used. Specifically, viral promoters such as the SV40-derived initial promoter and cytomegalovirus LTR, mammalian constitutive protein gene promoters such as the β-actin gene promoter, and RNA promoters such as the tRNA promoter, and the like are used.

When the expression of an siRNA or a micro RNA is intended, it is general that a pol III promoter be used as the promoter. As examples of the pol III promoter, the U6 promoter, H1 promoter, tRNA promoter and the like can be mentioned.

The above-mentioned expression vector preferably comprises a transcription termination signal, i.e., a terminator region, downstream of the above-described polynucleotide or nucleic acid that encodes the same. Furthermore, a selection marker gene for selection of transformant cells (a gene that confers resistance to a drug such as tetracycline, ampicillin, or kanamycin, a gene that compensates for an auxotrophic mutation and the like), and fluorescent protein genes (GFP, RFP, YFP etc.) can further be contained.

Although there is no limitation on the choice of expression vector used in the present invention, suitable vectors for administration to mammals such as humans include plasmid vector; viral vectors such as retrovirus, adenovirus, adeno-associated virus and lentivirus. Adenovirus, in particular, has advantages such as very high gene transfer efficiency and transferability to non-dividing cells as well. However, since the integration of transgenes into host chromosomes is extremely rare, the gene expression is transient and normally persists only for about 4 weeks. Considering the persistence of effect, it is also preferable to use adeno-associated virus and lentivirus, which offer a relatively high efficiency of gene transfer, which can be transferred into non-dividing cells as well, and which can be integrated into chromosomes.

Examples of the low molecular p38 inhibitor include, but not particularly limited to, pyridinylimidazoles, substituted pyrazoles, substituted pyridyls, quinazoline derivatives, aryl ureas, heteroaryl analogues, substituted imidazole compounds and substituted triazole compounds.

Many compounds have heretofore been proposed for p38 inhibition. The compounds proposed for p38 inhibition include pyridinylimidazoles. See Young P. R. et al. (1997) J. Biol. Chem. 272, pages 12116-12121; and Bender, P. E. (1985) J. Med. Chem. 28, pages 1169-1177. Examples of the pyridinylimidazoles capable of inhibiting p38 include 6-(4′-fluorophenyl)-5-(4′-pyridyl)-2,3-dihydroimidazo(2,1-b)-thiazole and a metabolite thereof (sulfoxide, sulfone), analogue thereof, fragment thereof and mimic thereof. Furthermore, it is proposed that 4-(pyridin-4-yl)-5-phenylimidazole, the smallest structure of the pyridinylimidazoles, could be sufficient to inhibit p38. See Gallagher, T F et al. (1997) Bio-org. Med. Chem. 5, pages 49-64.

Particular 1,5-diaryl substituted pyrazole compounds are also proposed as a p38 inhibitor. Such substituted pyrazole compounds are disclosed in U.S. Pat. No. 6,509,361. Further pyrazole derivatives inhibiting p38 are disclosed in U.S. Pat. No. 6,335,336.

Examples of other p38 inhibitors include substituted pyridyls such as those disclosed in US-B-2003/0139462, and the like.

Further p38 inhibitor are those disclosed in U.S. Pat. No. 6,610,688.

A quinazoline derivative can also function as a p38 inhibitor. Examples of the quinazoline derivative as the p38 inhibitor are disclosed in U.S. Pat. Nos. 6,541,477, 6,184,226, 6,509,363 and 6,635,644.

Aryl ureas and heteroaryl analogues can also function as p38 inhibitors. Examples of the aryl ureas and heteroaryl analogues as the p38 inhibitors are disclosed in U.S. Pat. No. 6,344,476. WO 99/32110 describes hetero ring-ureas as the p38 inhibitor. WO 99/32463 describes urea compounds inhibiting p38 kinase. WO 98/52558 describes urea compounds for p38 kinase inhibition. WO 99/00357 describes a method for using urea compounds as a p38 kinase inhibitor. WO 99/58502 describes urea compounds as a p38 kinase inhibitor.

A substituted imidazole compound and a substituted triazole compound can also function as p38 inhibitors. Such compounds are respectively disclosed in U.S. Pat. Nos. 6,560,871 and 6,599,910.

Examples of further p38 inhibitor include RWJ-67657 (RW Johnson Pharmaceutical Research Institute); RDP-58 (Sangstat Medical); RDP-58; Scios-469 (Solos); MKK3/MKK6 inhibitor (Signal Research Division); SB-210313 analogue, SB-220025, SB-238039, HEP-689, SB-203580, SB-239063, SB-239065, SB-242235 (SmithKline Beecham Pharmaceuticals); VX-702 and VX-745 (Vertex Pharmaceuticals); AMG-548 (Amgen); Astex p38 kinase inhibitor (Bayer); BIRB-796 (Boehringer Ingelheim Pharmaceuticals); Celltech p38 MAP kinase inhibitor (CeltechGroup plc.); FR-167653 (Fujisawa Pharmaceutical); 681323 and SB-281832 (GlaxoSmithKline plc); MAP kinase inhibitor of LEO Pharmaceuticals (LEO Pharma A/S); Merck&Co. p38 MAP kinase inhibitor (Merck research Laboratories); SC-040 and SC-XX906 (Monsanto); Novartis adenosine A3 antagonists (Novartis AG); p38 MAP kinase inhibitor (NovartisPharma AG); CP-64131 (Pfizer); CNI-1493 (Picower Institute for Medical Research); RPR-200765A (Phone-Poulenc Rorer); and Roche p38 MAP kinase inhibitor and Ro-320-1195 (Roche Bioscience).

In one embodiment, a low molecular p38 inhibitor is SB203580.

The agent for promoting neuronal differentiation of the present invention can contain, in addition to a p38 inhibitor, any carrier, for example, a pharmaceutically acceptable carrier.

Examples of the pharmaceutically acceptable carrier include, but are not limited to, excipients such as sucrose, starch, mannit, sorbit, lactose, glucose, cellulose, talc, calcium phosphate, calcium carbonate and the like, binders such as cellulose, methylcellulose, hydroxypropylcellulose, polypropylpyrrolidone, gelatin, gum arabic, polyethylene glycol, sucrose, starch and the like, disintegrants such as starch, carboxymethylcellulose, hydroxypropylstarch, sodium-glycol-starch, sodium hydrogen carbonate, calcium phosphate, calcium citrate and the like, lubricants such as magnesium stearate, Aerosil, talc, sodium lauryl sulfate and the like, aromatics such as citric acid, menthol, glycyrrhizin•ammonium salt, glycine, orange powder and the like, preservatives such as sodium benzoate, sodium bisulfite, methylparaben, propylparaben and the like, stabilizers such as citric acid, sodium citrate, acetic acid and the like, suspensions such as methylcellulose, polyvinylpyrrolidone, aluminum stearate and the like, dispersing agents such as surfactant and the like, diluents such as water, saline, orange juice and the like, base waxes such as cacao butter, polyethylene glycol, white petroleum oil and the like, and the like.

When a nucleic acid which hybridizes, under physiological conditions, to a DNA or mRNA encoding p38, thereby inhibiting the transcription and/or translation thereof, or an expression vector thereof is used as a p38 inhibitor, the agent for promoting neuronal differentiation of the present invention can further contain a reagent for nucleic acid introduction, to promote introduction of the nucleic acid into cells. As the reagent for nucleic acid introduction, a cationic lipid such as lipofectin, lipofectamine, lipofectamine RNAiMAX, Invivofectamine, DOGS (Transfectam), DOPE, DOTAP, DDAB, DHDEAB, HDEAB, polybrene, poly(ethyleneimine) (PEI) and the like can be used. When retrovirus is used as an expression vector, RetroNectin, fibronectin, polybrene and the like can be used as a reagent for introduction.

Examples of the dosage unit form of the agent of the present invention include liquid, tablet, pill, drinking liquid, powder, suspension, emulsion, granules, extract, fine granules, syrup, infusion, decoction, eye drop, troche, poultice, liniment, lotion, ophthalmic ointment, plaster, capsule, suppository, enema, injection (liquid, suspension and the like), adhesive preparation, ointment, jelly, paste, inhalant, cream, spray, nasal drop, aerosol and the like.

The content of the p38 inhibitor in the pharmaceutical composition is not particularly limited and is appropriately determined in a wide range. For example, it is about 0.01 to 100 wt % of the whole pharmaceutical composition.

The agent of the present invention can be used for promoting neuronal differentiation of neural stem cells. In the present specification, the neural stem cell refers to a cell having an ability to differentiate into any type of cell constituting the central nervous system (neural multipotency), including neuron, astrocyte and oligodendrocyte, and having self replication potency. Neural stem cells generated from pluripotent stem cells such as fertilized eggs, embryonic stem cells, induced pluripotent stem cells and the like temporally changes their responsiveness to extrinsic differentiation-inducing stimulation while maintaining the multipotency. To be specific, in early neural stem cells, the neurogenic competence to differentiate into neurons in response to neurogenic signals is dominant, and the gliogenic competence to differentiate into astrocytes in response to gliogenic signals is recessive. However, along with the progress of differentiation stages, the neurogenic competence disappears, the gliogenic competence are acquired, and the cells transit into late neural stem cells wherein neurogenic competence is recessive and gliogenic competence is dominant. The present invention has been made based on the findings that p38 is involved in the transition of neural stem cells where neurogenic competence is dominant than gliogenic competence (neurogenic neural stem cell) into neural stem cells where gliogenic competence is dominant than neurogenic competence (gliogenic neural stem cell), specifically that p38 is involved in the acquisition of gliogenic competence of the neural stem cells, and expression of p38 is suppressed by micro RNA such as miR-17 and the like in the early neural stem cells.

In the present specification, promotion of neuronal differentiation of neural stem cells means to increase neurogenic competence and decrease gliogenic competence of neural stem cells, or as a result of which enhance differentiation of neural stem cell into neuron. In one embodiment, the neural stem cells used in the present invention are gliogenic. When a p38 inhibitor is applied to the gliogenic neural stem cells, neurogenic competence increases and gliogenic competence is suppressed. As a result, conversion of gliogenic neural stem cells to neurogenic neural stem cells occurs. In one embodiment, the neural stem cells used in the present invention are neurogenic. When a p38 inhibitor is applied to the neurogenic neural stem cells, both a decrease in the neurogenic competence and acquisition of gliogenic competence are suppressed, and the neurogenic stage is maintained. In the present invention, both of these embodiments are encompassed in the promotion of neuronal differentiation of neural stem cells.

Whether the neural stem cells are neurogenic or gliogenic can be evaluated by culturing the evaluation target neural stem cells in the presence of glial differentiation inducing factors, LIF and BMP2, according to the method described in Nat. Neurosci. 11, 1014-1023 (2008), and examining which of the neurons and astrocytes are generated in a greater number. When the number of the astrocytes is higher than that of the neurons as a result of the culture, the neural stem cells are judged to be gliogenic; otherwise, the neural stem cells can be judged to be neurogenic.

In the present invention, the neural stem cells may be present in vivo or in vitro. Therefore, the agent for promoting neuronal differentiation of the present invention can be used as an in vivo medicament or a reagent for experiment in vitro.

Since the neural stem cells are present in vivo in the cerebral cortex in the embryonic stage/fetal stage, and hippocampus and lateral cerebral ventricle of adult, in human or non-human mammal (e.g., mouse), a p38 inhibitor (or the agent for promoting neuronal differentiation of the present invention) is administered to an individual mammal such that it is delivered to the neural stem cells in the neural tissues in the body, whereby neuronal differentiation of neural stem cells can be promoted in vivo.

While the disease to be the target of medicament is not particularly limited as long as it is a disorder for which promotion of neuronal differentiation of neural stem cells is required in human or non-human mammal, examples of such disorder include nerve system diseases and nerve injuries in the central nervous system, such as cerebral infarction, traumatic brain injury, cerebral ischemia, Huntington's disease, Alzheimer's disease, functional disorder due to aging and the like.

When in use, the agent for promoting neuronal differentiation of the present invention is administered according to a method suitable for each form. For example, in the case of an external preparation, the agent is sprayed, adhered or applied directly onto the required site such as skin, mucosa and the like; in the case of tablet, pill, drinking liquid, suspension, emulsion, granule or capsule, the agent is administered orally; in the case of injection, the agent is administered intravenously, intracerebrally, intramuscularly, intradermally, subcutaneously, intraarticularly or intraperitoneally; and in the case of suppository, the agent is administered intrarectally.

While the dose of the agent of the present invention varies depending on the activity and choice of the active ingredient, administration manner (e.g., oral, parenteral), recipient animal species, the recipient's drug receptivity, body weight, age, and the like, and cannot be generalized, the dose is normally about 0.001 mg to about 2.0 g, based on the amount of active ingredient, per day for an adult.

Moreover, when the agent for promoting neuronal differentiation of the present invention is used in vitro, the neuronal differentiation of the neural stem cells can be promoted by cultivating the neural stem cells in the presence of a p38 inhibitor (or the agent for promoting neuronal differentiation of the present invention). The present invention also provides such method of promoting neuronal differentiation of neural stem cells.

While the neural stem cells in a culture state may be obtained by isolating neural stem cells in the living body and culturing same, they may also be prepared by differentiating pluripotent stem cells such as embryonic stem cells (ES cell), induced pluripotent stem cells (iPS cell) and the like into the neural stem cells. A method of inducing differentiation of ES cells into neural stem cells is disclosed in detail in JP-A-2002-291469 and the like. The method is also applicable to other pluripotent stem cells such as iPS cell and the like.

To enhance differentiation potency of pluripotent stem cells into the nerve system in the differentiation induction into neural stem cells, it is preferable to form embryoid (embryoid body; EB) in advance and differentiate the EB into neural stem cells under particular culture conditions. EB can be formed by cultivating ES cells or iPS cells in the presence of, for example, retinoic acid or Noggin protein. In the case of retinoic acid, it may be added to a medium at a low concentration level (10⁻⁹ M-10⁻⁶ M). In the case of Noggin protein, a culture supernatant obtain by introducing Xenopus Noggin into mammalian cultured cells, and allowing for transient expression of the Noggin protein may be directly (1-50% (v/v)) used, or a recombinant Noggin protein (final concentration about 1 μg/ml) may also be used.

The thus-obtained EB is dissociated and subjected to suspension culture in a serum-free medium added with FGF-2 (generally 10-100 ng/ml), whereby neural stem cells can be obtained in the form of neurosphere. While the serum-free medium is not particularly limited as long as it is a serum-free medium generally used for culturing neural stem cells or neurons, examples thereof include a DMEM medium added with, besides the above-mentioned components, glucose, glutamine, insulin, transferrin, progesterone, putrescine, selenium chloride, heparin and the like. The medium to be used for neurosphere formation may be added with a sonic hedgehog protein (generally, 1-20 nM). Since addition of a sonic hedgehog protein improves differentiation induction efficiency of neural stem cells into neural progenitor cells and growth efficiency thereof, the differentiation efficiency into neurons by differentiation culture thereafter is enhanced. The culture is preferably performed under 5% CO₂ conditions at 35-40° C. for about 7-9 days.

In this case, a neurosphere directly derived from EB is referred to as a primary neurosphere, and a neurosphere reformed by separating and dispersing the primary neurosphere and cultivating same under the same conditions is referred to as a secondary neurosphere. In the following, a neurosphere passaged even only once under culture conditions is referred to as a secondary neurosphere. Generally, neural stem cells contained in the primary neurosphere are neurogenic. Neural stem cells contained in the secondary neurosphere generally have lower neurogenic competence and higher gliogenic competence as compared to the neural stem cells contained in the primary neurosphere. In one embodiment, neural stem cells contained in the secondary neurosphere are gliogenic.

When the thus-obtained neurosphere containing neural stem cells is cultivated under nerve cell differentiation conditions, differentiation of the neural stem cells into neurons is induced. The neuronal differentiation is promoted by the addition of a p38 inhibitor (or the agent for promoting neuronal differentiation of the present invention) in the neurosphere stage. When a nucleic acid which hybridizes, under physiological conditions, to a DNA or mRNA encoding p38, thereby inhibiting the transcription and/or translation thereof, or an expression vector thereof is used as a p38 inhibitor, a reagent for nucleic acid introduction may also be used to promote introduction of the nucleic acid into cells. While the neuronal differentiation conditions are not particularly limited, for example, culture in a medium free of mitogen such as FGF-2 and the like, and the like can be mentioned. As the medium, conventional media applicable for culturing neural stem cells and neurons can be used as long as mitogen is not contained. Examples thereof include DMEM:F12 medium containing glucose, glutamine, insulin, transferrin, progesterone, putrescine, selenium chloride, and the like. Sonic hedgehog protein may be present or absent. Examples of other neuronal differentiation conditions include those described in Nat Biotechnol. 21, 183-186 (2003) and the like, and those of ordinary skill in the art can easily construct various neuronal differentiation conditions. A differentiation culture into neuron is preferably performed under 5% CO₂ conditions at 35-40° C. for 5-7 days. When a primary neurosphere is used as a neurosphere, the p38 inhibitor acts on neurogenic neural stem cells contained therein, and both a decrease in the neurogenic competence and acquisition of the gliogenic competence are suppressed and the neurogenic stage of the neural stem cells is maintained. As a result, differentiation of neural stem cells into neurons is promoted. When a secondary neurosphere is used as a neurosphere, the p38 inhibitor acts on neural stem cells having the gliogenic competence (preferably gliogenic neural stem cells) contained therein to increase the neurogenic competence and suppress the gliogenic competence. As a result, restoration of differentiation potency is occurred into a neurogenic neural stem cell and differentiation of neural stem cells into neurons is promoted.

The contents disclosed in any publication cited in the present specification, including patents and patent applications, are hereby incorporated in their entireties by reference, to the extent that they have been disclosed herein.

The present invention is explained in more detail in the following by referring to the Examples shown below, which are not to be construed as limitative.

### EXAMPLES

### <Material and Method>

### Cell Culture and Neurosphere Differentiation Assay

Mouse ESC (EB3) culture, EB formation, neurosphere formation and differentiation of neurosphere were performed as previously described (Nat. Neurosci. 11, 1014-1023 (2008)). Human NSCs were grown as previously described (J. Neurosci. Res. 69, 869-879 (2002); J. Neurosci. Res. 87, 307-317 (2009)) without epithelial growth factor (EGF), and allowed to differentiate on a substrate-coated coverslips in the absence of mitogen for 14 days, a procedure similar to that used for mouse neurosphere.

### Lentivirus Preparation

Lentivirus particles were produced by the transient transfection of 293T human embryonic kidney cells with a lentiviral expression construct for the gene of interest, along with two lentivirus constructs required for viral replication (pCMV-VSV-G-RSV-Rev and pCAG-HIVgp). The transfection was performed using GeneJuice (Novagen) according to the manufacturer's instructions. The virus particles were collected by ultracentrifugation and then resuspended in phosphate-buffered saline. High-titer viral suspensions were used to obtain an efficient infection of NSCs with a multiplicity of infection of about 5.

### Animal and Uterus Virus Infection

Experiments were performed using ICR mice. All aspects of animal care and treatment were carried according to the guidelines of the Experimental Animal Committee of Keio University School of Medicine. In utero microinjection of lentivirus particles into the brain of E10.5 ICR mouse was guided by an in vivo ultrasound real-time scanner (Vevo660; VisualSonics).

### Immunostaining

Immunocytechemistry and immunohistochemistry were performed as previously described (Nat. Neurosci. 11, 1014-1023 (2008)) using antibodies against βIII-tubulin, NeuN (neural marker), glial fibrillary acidic protein (GFAP, astrocyte marker), and haemagglutinin (HA).

### Proteomics Analysis

Quantitative proteomics analyses using an iTRAQ method were performed for miR-LacZ-, and miR-17s-overexpressing p0 and p2 neurospheres, using AB SCIEX TripleTOF 5600 System (AB SCIEX) according to the manufacturer's instructions.

### Statistical Analysis

At least three independent experiments were performed for each statistical analysis. The statistical significance was determined by two-tailed t-test. Throughout the study, P<0.05 was considered to be significantly different.

### <Results>

### Identification of Genes Acting Downstream of Coup-tfs

The present inventors began this experiment by identifying candidate genes at the downstream of Coup-tfs. The direct DNA-binding sites of Coup-tfs were investigated using chromatin immunoprecipitation sequencing (ChIP-seq), and global gene and microRNA expression profiles were compared between control and Coup-tfs knockdown mouse neurospheres by microarray analysis (Nat. Neurosci. 11, 1014-1023 (2008)). Based on the obtained data, candidate genes were acquired and lentiviral libraries were constructed for their overexpression and knockdown. Next, the present inventors used the neurosphere culture system to functionally screen the candidate genes as previously described (Nat. Neurosci. 11, 1014-1023 (2008)). Finally, the miR-17-92 microRNA gene cluster was identified as a downstream effector of Coup-tfs.

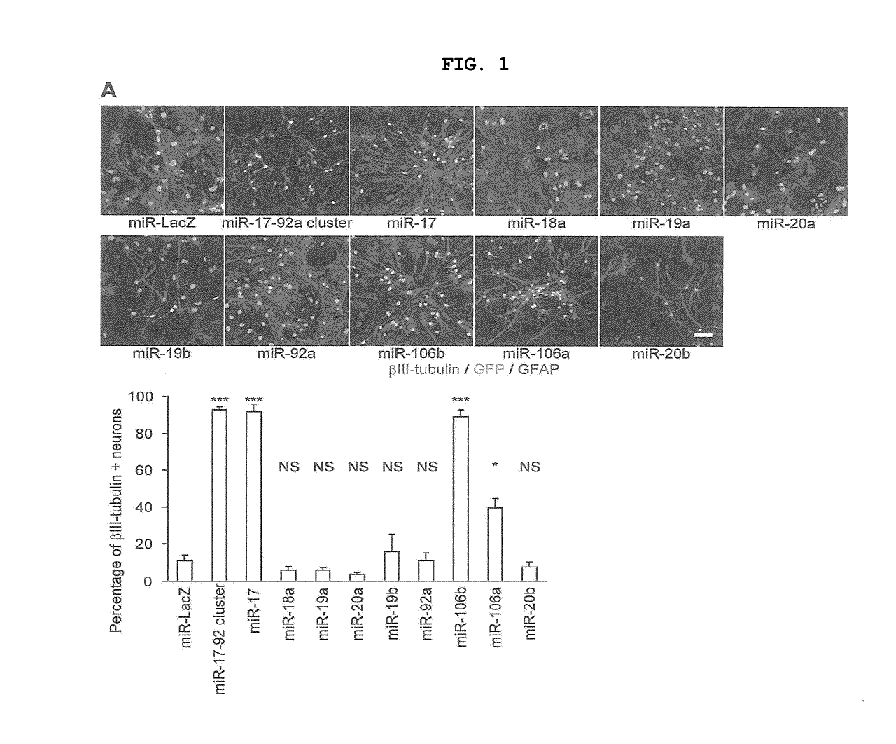

The miR-17-92 cluster was first identified as a cluster of oncogenic microRNAs, namely OncomiR-1, and was associated with the development of various organs (Nature 435, 828-833 (2005); Cell 133, 217-222 (2008); Cell 132, 875-886 (2008)). According to microarray analysis, the expression of this gene cluster was 3.58-fold higher in twice-passaged (p2) Coup-tfs knockdown neurospheres than in control neurospheres. Normally, the majority of cells in p2 neurospheres differentiate into astrocytes, with less than 20% becoming neurons. However, overexpression of the miR-17-92 cluster caused them to differentiate almost exclusively into neurons, with the elaboration of long βIII-tubulin-positive processes (FIG. 1).

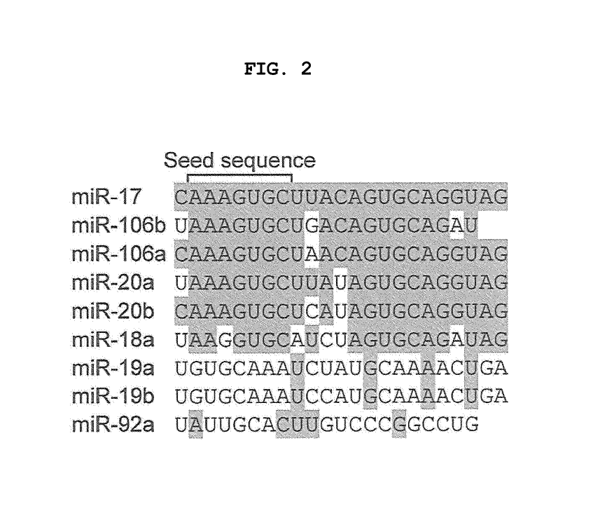

The miR-17-92 cluster encodes six distinct microRNAs (miR-17, 18a, 19a, 20a, 19b and 92a, with their star (*) strands). To determine which of the six microRNAs was responsible for the neuronal phenotype, the present inventors individually overexpressed each of them in NSCs. This was done by infecting NSCs with lentiviruses expressing microRNAs and green fluorescent protein (GFP). Of the six microRNAs, only miR-17 replicated the phenotype induced by the intact miR-17-92 cluster (FIG. 1). The nucleotide sequences of miR-17 and miR-20a are highly homologous and share the same 7-mer seed sequence (nucleotides 2-8, FIG. 2) that is required for binding to target mRNAs. Therefore, the target mRNAs of these two microRNAs are also speculated to have highly overlapping sequences (Cancer Res. 68, 8191-8194 (2008)). However, the overexpression of miR-20a did not yield a neuronal promoting action (FIG. 1).

MiR-17 has two additional paralogs with high homology and identical seed sequences, miR-106b and miR-106a, which are encoded by the miR-106b-25 and miR-106a-363 clusters, respectively (Cancer Res. 68, 8191-8194 (2008)). These paralogs were individually overexpressed in NSCs to examine whether either or both affected the neurogenesis-to-gliogenesis switch. Overexpression of both microRNAs led to a neuronal phenotype, although the effect of miR-106b was stronger than that of miR-106a (FIG. 1). Thus, miR-17, miR-106a and miR-106b were collectively termed “miR-17s” in Examples of the description.

As demonstrated previously, knockdown of Coup-tfs causes sustained generation of Islet-1 (Isl-1)-expressing neurons, a subpopulation of early-born neurons found even at the p2 neurosphere stage (Nat. Neurosci. 11, 1014-1023 (2008)). Notably, overexpression of miR-17s did not result in the cytogenesis of Isl-1-positive neurons, even though miR-17s were identified as downstream effectors of Coup-tfs. Instead, Onecut2 was identified as a positive regulator of Isl-1-positive cell production during candidate gene screening. These results suggested that both neurogenesis-to-gliogenesis transition in NSCs and neuron subtype specification are independently regulated downstream of Coup-tfs.

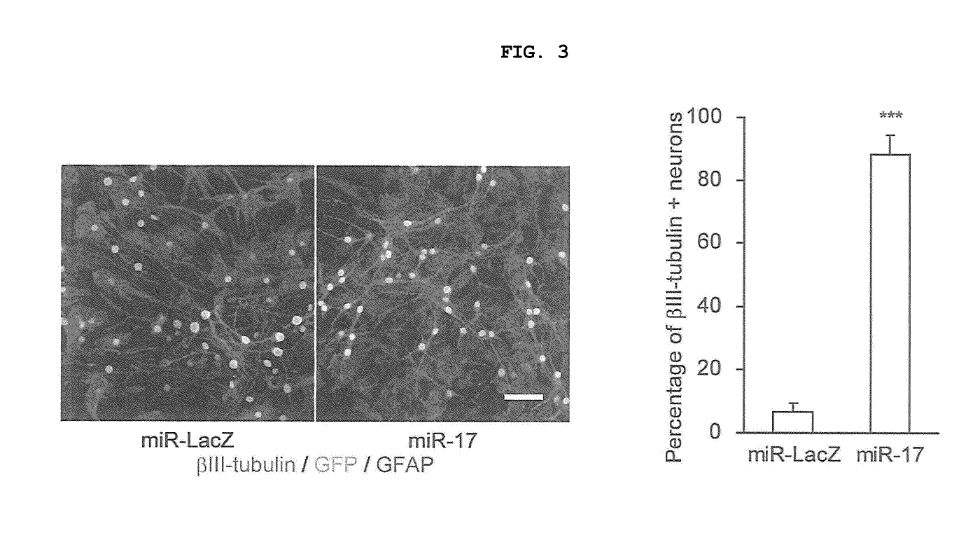

The transition from early-developmental neurogenic NSCs to late-developmental gliogenic NSCs can be recognized by changes in the responsiveness of NSCs to gliogenic cytokines (Nat. Neurosci. 11, 1014-1023 (2008)). LIF and BMP2 are well-known extrinsic glial differentiation inducing factors that promote gliogenesis only in late-developmental NSCs by activating the JAK-STAT pathway and BMP signaling, respectively (Proc. Natl. Acad. Sci. U.S.A. 95, 3178-3181 (1998); Science 284, 479-482 (1999); Proc. Natl. Acad. Sci. U.S.A. 98, 5868-5873 (2001)). LIF does not act as a gliogenic factor in early-developmental NSCs because the STAT3-binding site in the promoter of glial marker genes is epigenetically suppressed (epigenetically silenced) by DNA methylation (Dev. Cell 1, 749-758 (2001)). BMP signaling, for its part, promotes neuronal rather than glial differentiation in early-developmental NSCs (Proc. Natl. Acad. Sci. U.S.A. 98, 5868-5873 (2001); J. Neurosci. 18, 8853-8862 (1998)). Therefore, to examine whether miR-17 alters the responsiveness of NSCs to glial differentiation induction cytokines, miR-17-overexpressing NSCs were exposed to LIF and BMP2 during differentiation. MiR-17-overexpressing neurospheres were strongly resistant to these cytokines and differentiated exclusively into neurons, even at the p2 stage (FIG. 3).

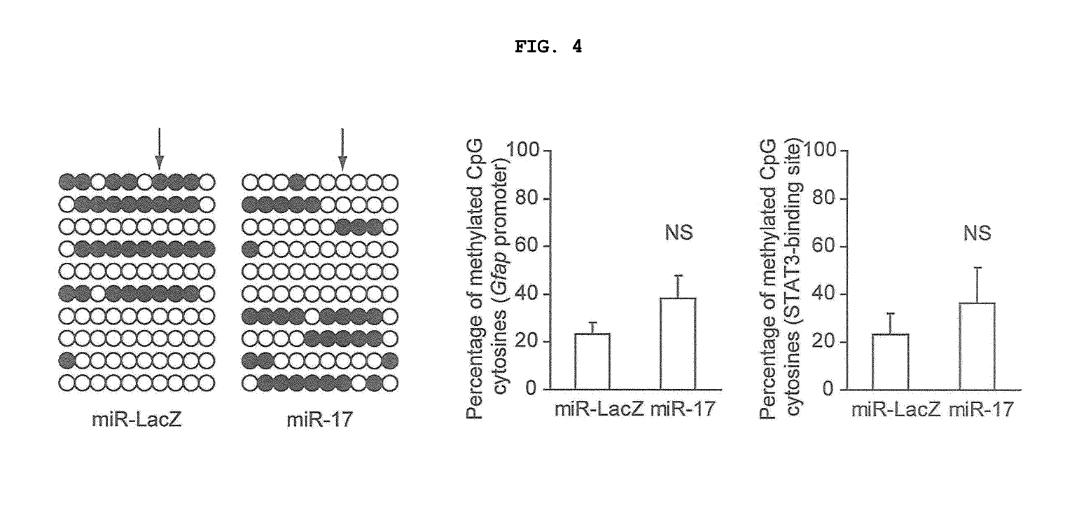

The present inventors next investigated the temporally-regulated DNA methylation status of the STAT3-binding site and its surrounding CpG sites in the Gfap promoter. Gfap is the most general gene marker for astrocytes. However, no significant changes in the extent of CpG methylation at the p2 neurosphere stage were observed following the overexpression of miR-17 (FIG. 4). Coup-tfs are transiently upregulated in developing NSCs during mid-gestation. After this time, NSCs lose their plasticity and produce only late-born subtype neurons and glial cells. Because knockdown of Coup-tfs in ESC-derived neurospheres results in the maintenance of the early epigenetic status of the Gfap promoter (Nat. Neurosci. 11, 1014-1023 (2008)), the present inventors initially expected that the expression of the molecular entities (e.g., miR-17s) responsible for the changes in NSC competence would converge with changes in the epigenetic status of neuronal or glial differentiation-associated genes. However, the current results suggest that miR-17s function at another stage in Coup-tfs-regulated competency transition. The change in the epigenetic status of cell-type specific genes may therefore be only one necessary prerequisite for the control of cytogenesis.

### MiR-17s as Determinants for the Neurogenic Competence of NSCs

To ensure that miR-17s are downstream effectors of Coup-tfs, the present inventors next performed experiments using RNA decoys, namely “tough decoy (TuD)” RNA, which strongly and stably suppresses specific target microRNAs (Nucleic Acids Res 37, e43 (2009)). Lentivirus vectors were constructed that expressed miR-17-specific TuD, miR-17*-specific TuD (TuD-miR-17 and TuD-miR-17*, respectively), or control TuD-miR-LacZ.

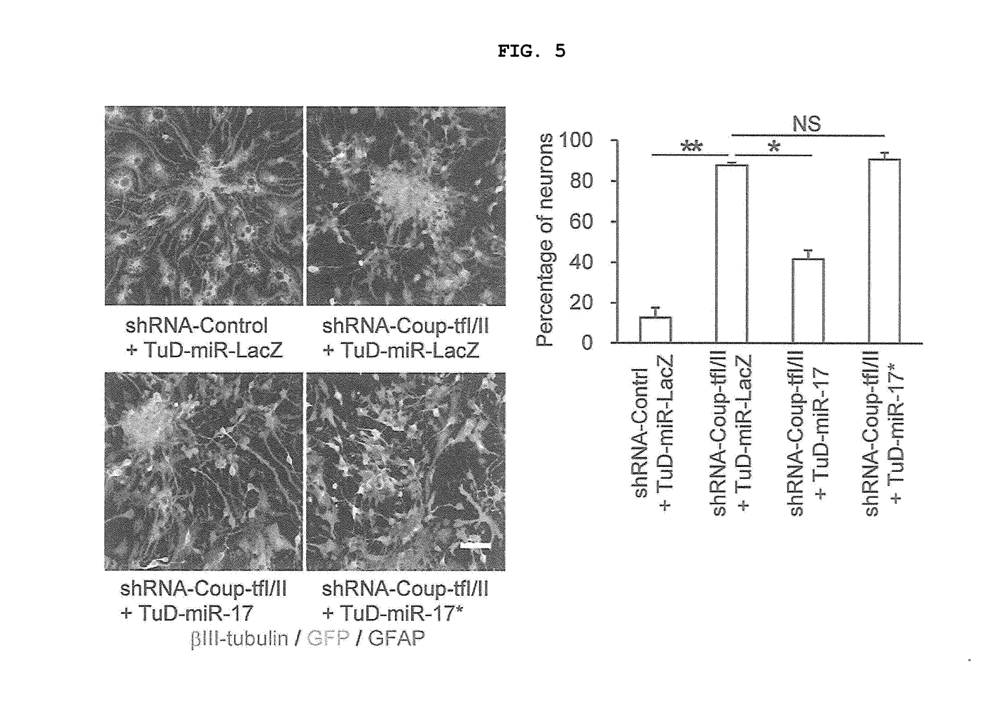

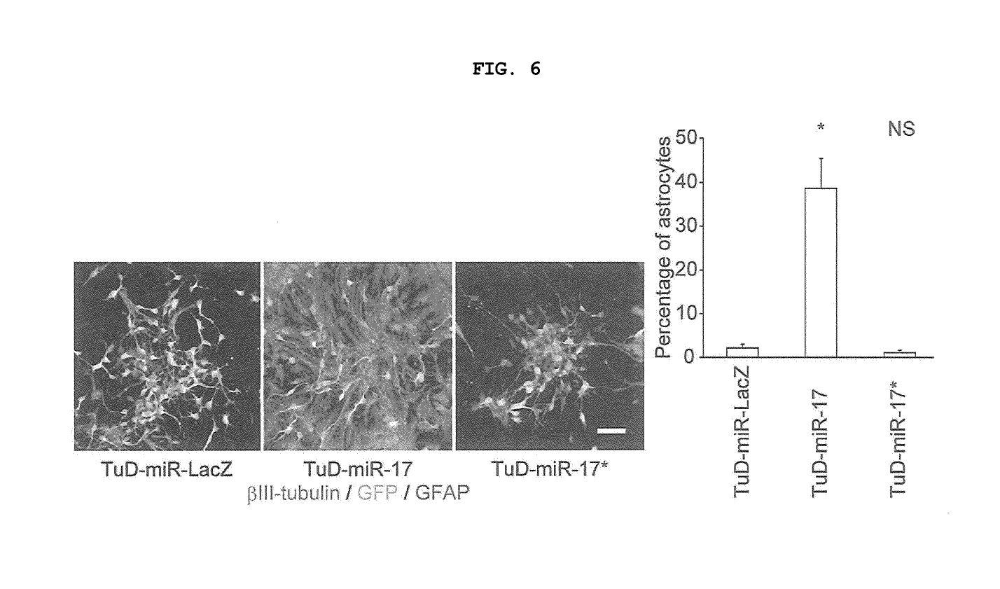

This series of experiments employed neurospheres in which Coup-tfs was silenced using Coup-tfs-specific short hairpin RNA (shRNA) from the p0 stage (Nat. Neurosci. 11, 1014-1023 (2008)). The neurogenesis-to-gliogenesis switch is inhibited in these suppressed (silenced) neurospheres, as it is in miR-17-overexpressing neurospheres. As expected, TuD-miR-17 expression largely cancelled out the neuronal differentiation promoting action induced by Coup-tfs knockdown at the p2 stage (FIG. 5), whereas the TuD-miR-17* and Tud-mi-17-LacZ controls had no significant effect. Moreover, while p0 neurospheres barely differentiate into glia, even in the presence of gliogenic cytokines, the forced expression of TuD-miR-17 (but not TuD-miR-17*) resulted in the induction of precocious gliogenesis, but only in the presence of LIF and BMP2 (FIG. 6). These results clearly demonstrate that miR-17s are responsible for the neurogenic competence of NSCs, at least in part due to their actions downstream of Coup-tfs.

Functions of miR-17s in the Developing CNS

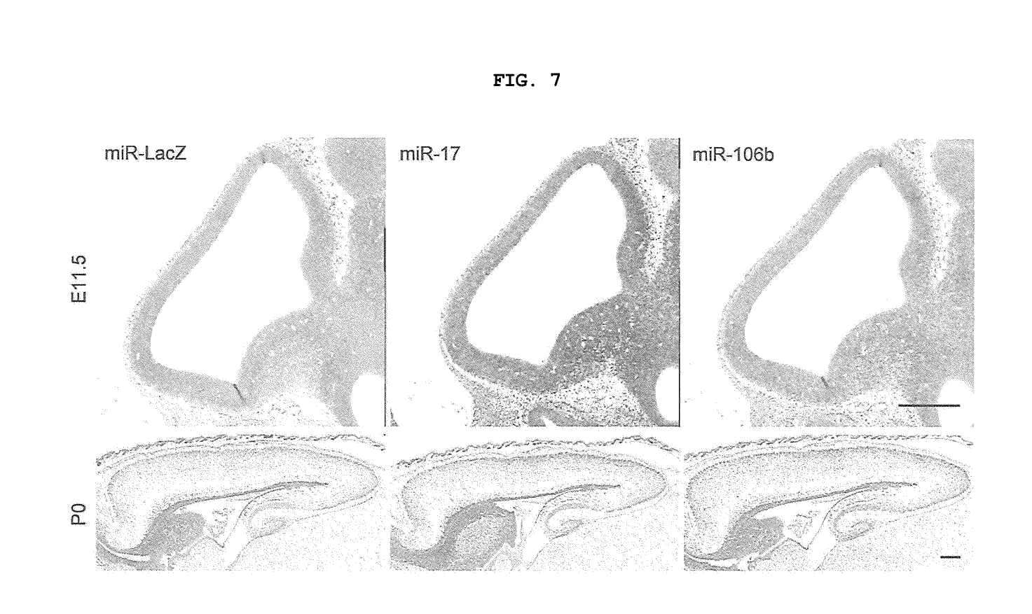

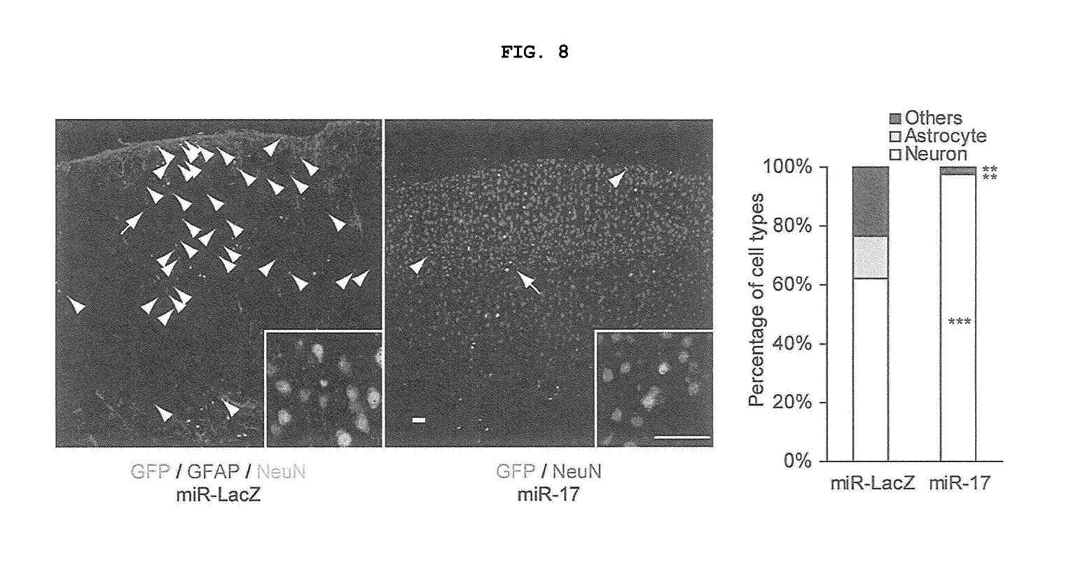

As a first step toward exploring the functional roles of miR-17s in vivo, the present inventors investigated their expression patterns in the developing CNS. According to the microarray data, expression levels of miR-17 and miR-106b were 0.18- and 0.28-fold lower in p2 neurospheres than in p0 neurospheres. As expected from these data, the in vivo expression patterns of miR-17 and miR-106b were similar at embryonic Day 11.5 (E11.5) and postnatal Day 0 (P0), although a stronger signal was observed for the former at E11.5. The levels of both microRNAs declined as development progressed (FIG. 7). Next, the in vivo roles of miR-17s were analyzed by examining the effects of miR-17 overexpression in the developing mouse brain via in utero lentiviral microinjection. Consistent with the in vitro results, most of the cells (97.6%) infected with the miR-17-overexpressing lentivirus at E10.5 were fated to become neurons in the cerebral cortex by P30, whereas only 62.2% of the cells infected with the control miR-LacZ-expressing lentivirus became neurons (FIG. 8). Thus, overexpression of miR-17 also inhibited gliogenesis in vivo in a cell-autonomous fashion.

Identification of p38 as a Direct Target for miR-17s

Normally, a microRNA regulates the degradation and/or translation of multiple target mRNAs (Biogerontology 11, 501-506 (2010)). To further identify the molecular mechanisms underlying changes in the competence of NSCs, the present inventors tried to identify the target mRNAs of miR-17s. To this end, the present inventors first carried out a proteomics analysis using the iTRAQ (isobaric tags for relative and absolute quantitation) method to identify proteins downregulated by the overexpression of miR-17 in p2 neurospheres (17). Candidate genes were then scanned using our original “in-house” computer program “HIMAJA”, which searches for sequences in mRNAs that are complementary to the seed sequences of particular microRNAs. An initial list of 40 genes was obtained. Finally, mRNA encoding p38 (also known as mitogen-activated protein kinase 14, or Mapk14) was identified as a direct target for miR-17s during the regulation of competence transition of NSCs as follows.

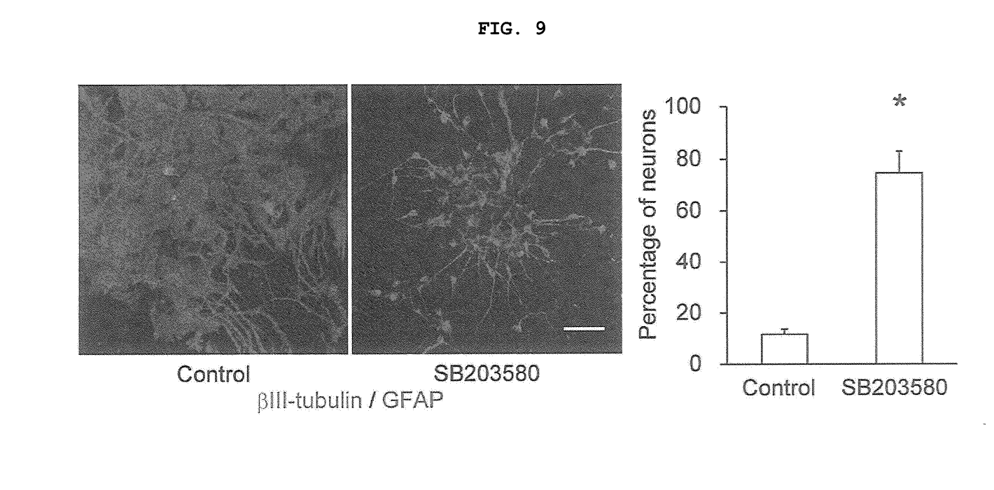

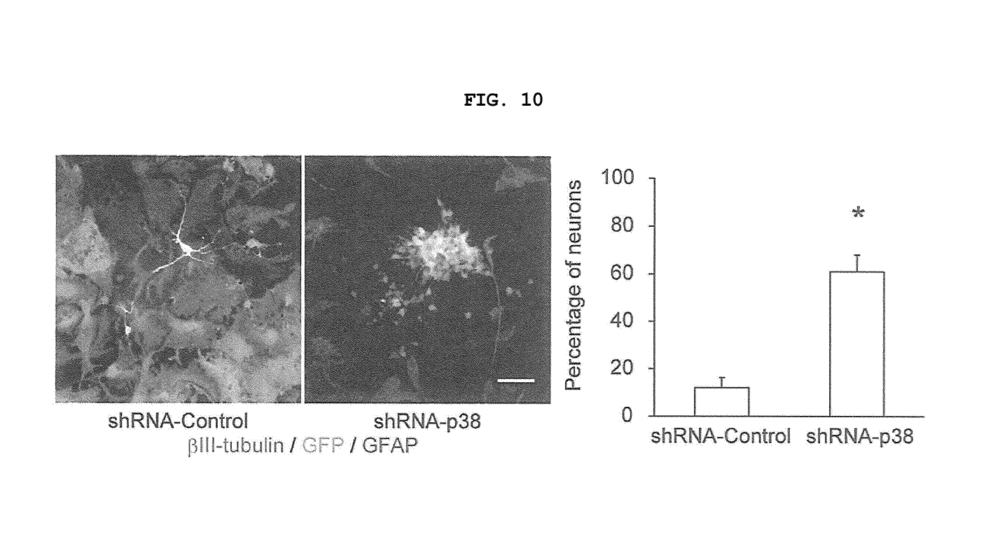

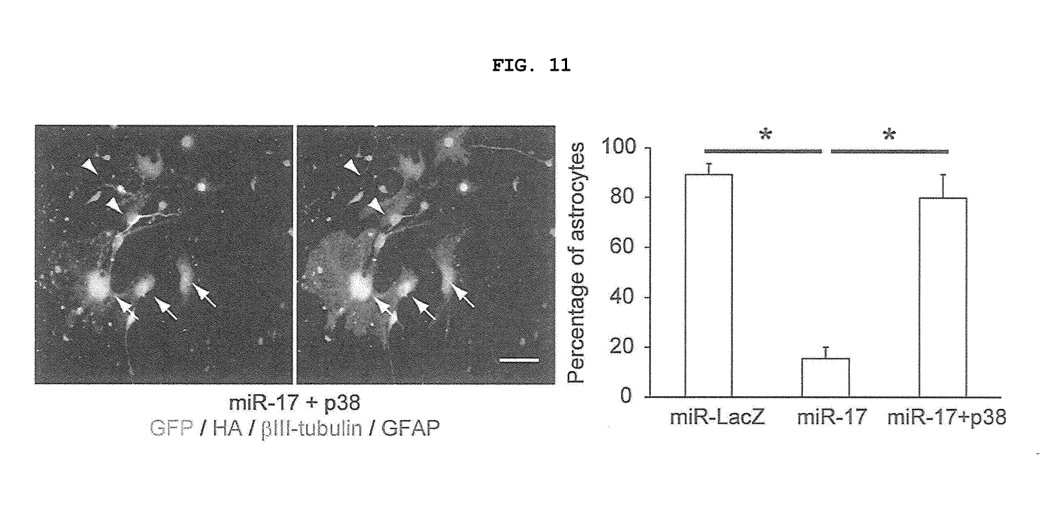

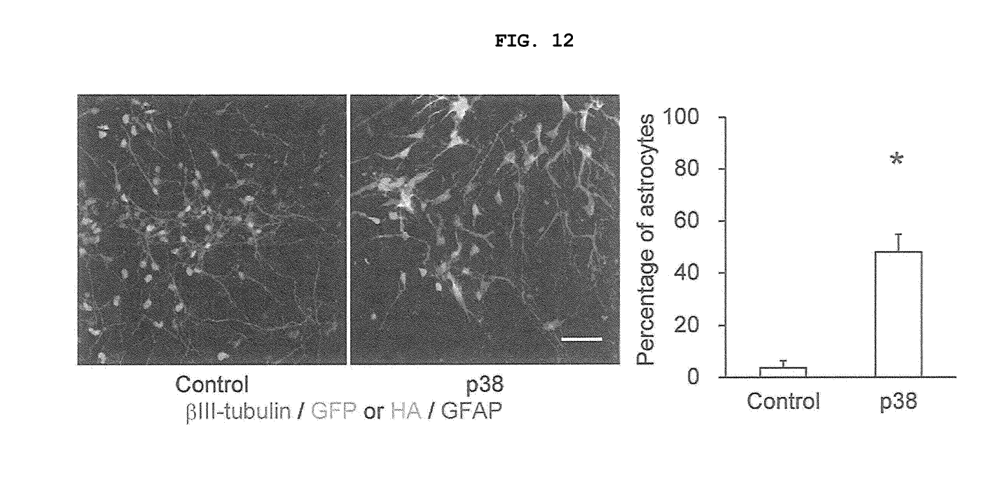

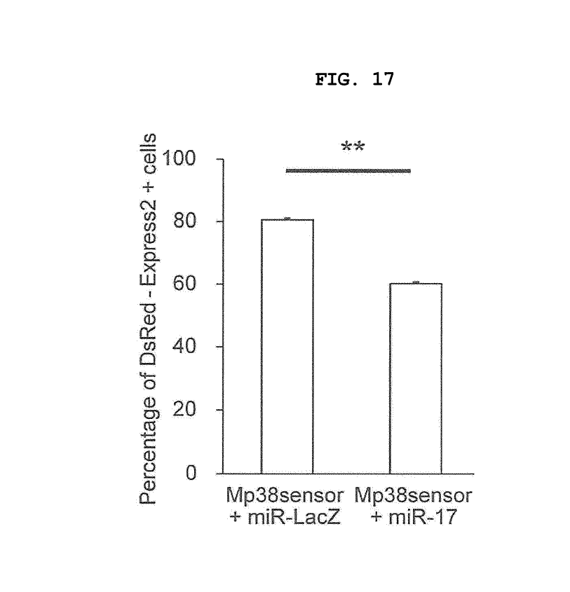

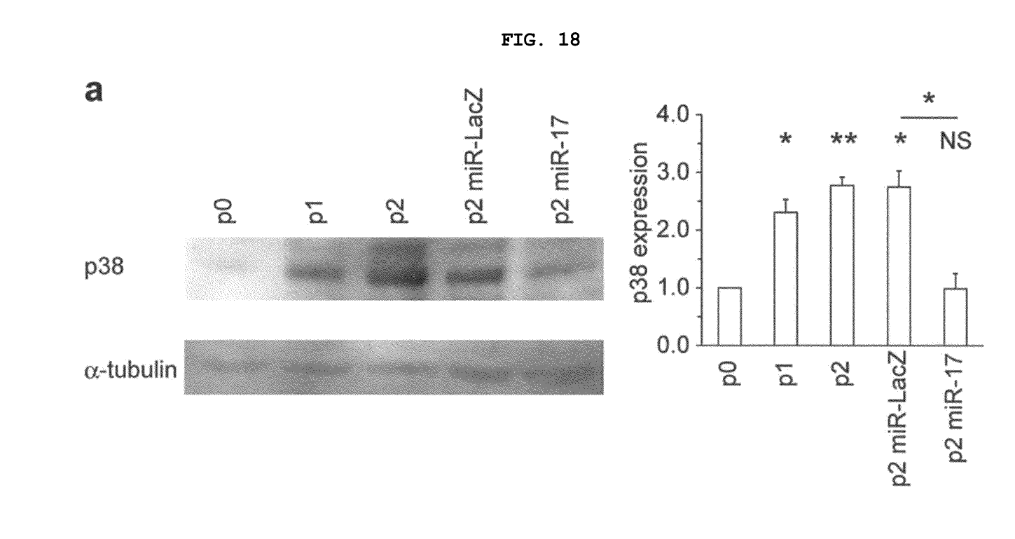

First, neurospheres at the p2 stage showed higher neuropotency than controls following exposure to a specific p38 inhibitor (SB203580) (FIG. 9), as did p2 neurospheres following knockdown of p38 using a p38-specific shRNA (FIG. 10). On the other hand, p38 protein expression increased during the passage of neurospheres (3.16-fold higher at p2 than at p0). In the Western blotting, the expression is 2.78-fold higher at p2 than at p0 (FIG. 18). Next, a reporter assay using the miR-17s-binding site in the 3′untranslated region (UTR) of p38 mRNA revealed a direct interaction between p38 mRNA and miR-17 (FIG. 17). Finally, the overexpression of miR-17s-resistant p38 mRNA, which lacks the entire 3′UTR, not only reversed the neuronal phenotype induced by miR-17 overexpression to a glial phenotype (FIG. 11) but also induced precocious gliogenesis from p0 neurospheres (FIG. 12). These results indicate that p38 is a direct target for miR-17s and is largely involved in the initiation of gliogenesis.

### Forced Restoration of Neuropotency in Gliogenic NSCs

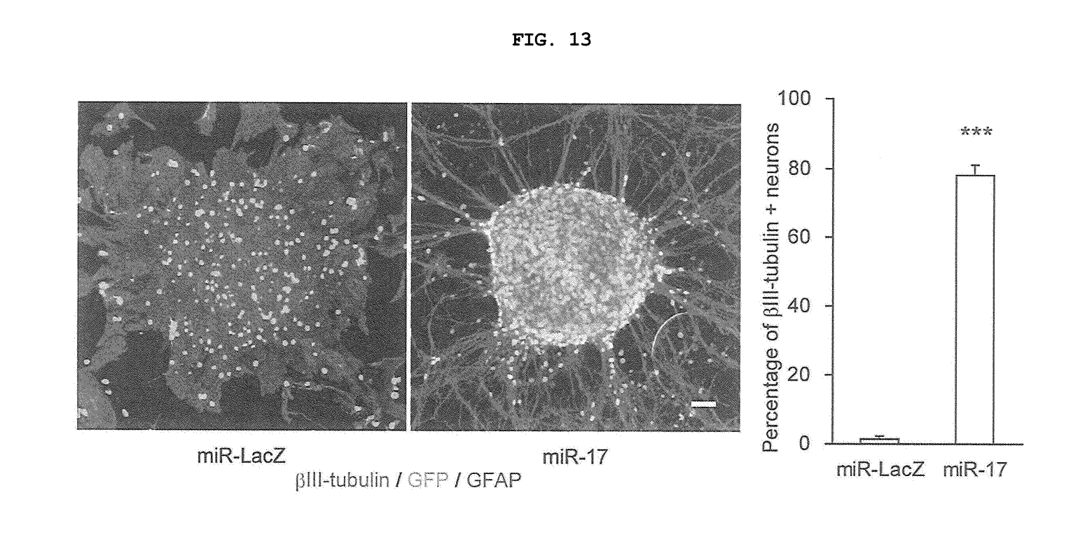

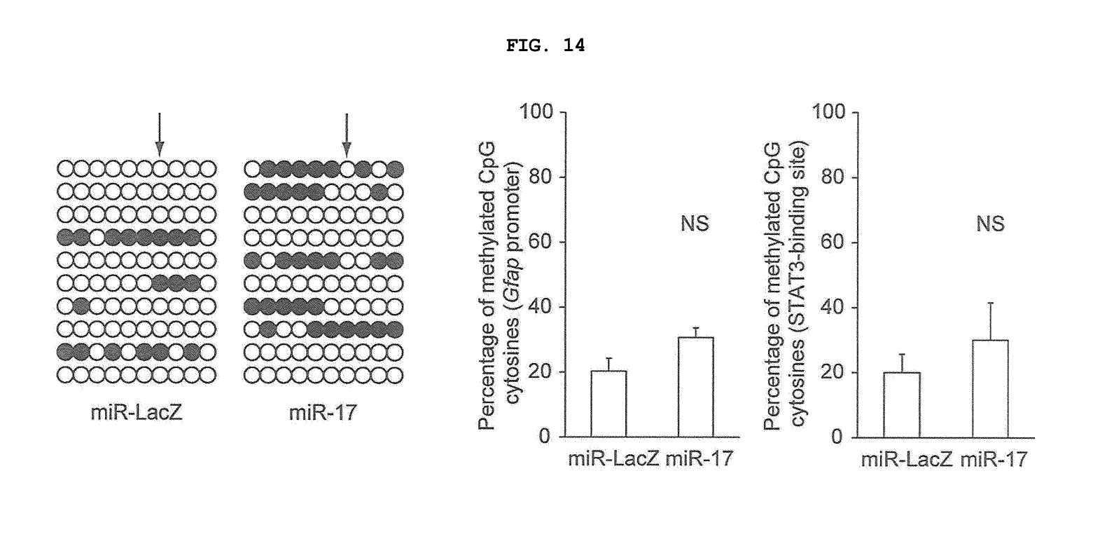

Finally, the present inventors asked whether the overexpression of miR-17s could restore neuropotency in stage-progressed NSCs. To address this, the present inventors overexpressed miR-17 in neurospheres via lentiviral infection at the onset of the p3 stage, when neurospheres normally differentiate exclusively into glial cells. Previous work of the present inventors showed that knockdown of Coup-tfs resulted in the recovery of neuropotency only in limited populations of p3 NSCs, which had apparently not yet lost their plasticity (Nat. Neurosci. 11, 1014-1023 (2008)). Surprisingly, however, overexpression of miR-17 led to a marked recovery of neuropotency in p3 neurospheres within 1 week, whereas the vast majority of cells within control p3 neurospheres differentiated into astrocytes (FIG. 13). In addition, the DNA methylation status of the Gfap promoter did not change significantly (FIG. 14), as observed previously during continuous upregulation of miR-17 in neurospheres from the p0 stage (FIG. 4).

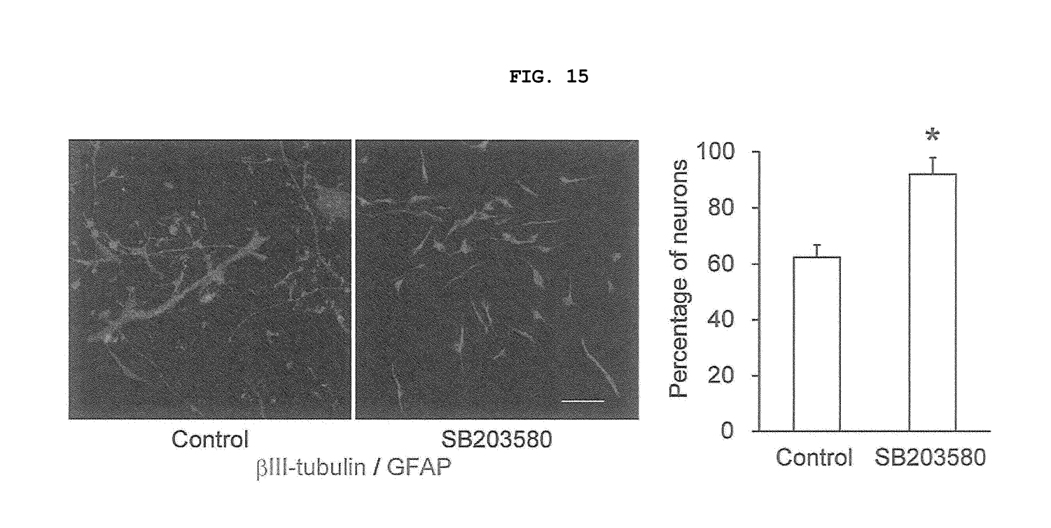

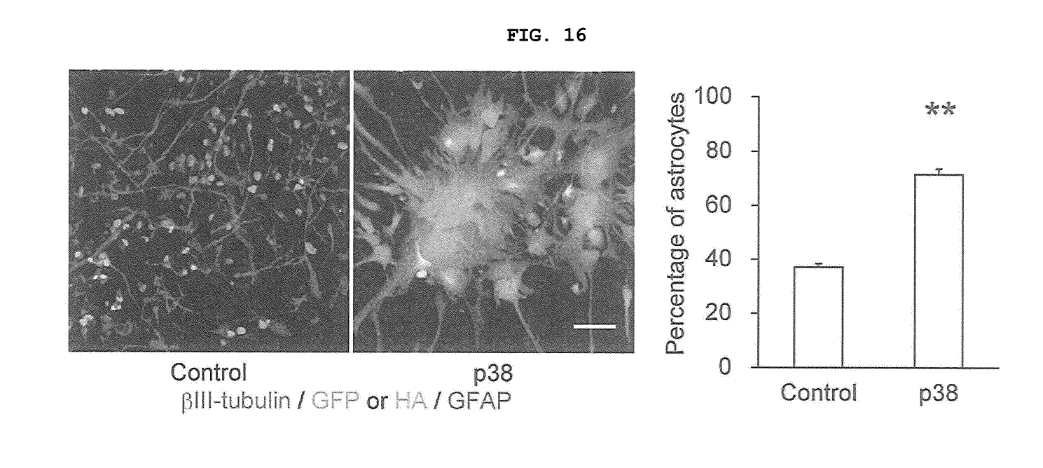

The specific p38 inhibitor, SB203580, and overexpression of p38 restored neuropotency (FIG. 15) and promoted gliogenesis (FIG. 16), respectively, in human fetal brain-derived neurospheres (J. Neurosci. Res. 69, 869-879 (2002); J. Neurosci. Res. 87, 307-317 (2009)). These results suggest that the molecular machinery directing the neuronal-to-glial competence transition of NSCs is evolutionarily conserved, but that its microRNA-mediated regulation differs among species.

### Functional Roll of p38 in Vivo in Developing Neural Stem/Progenitor Cells (NSPCs)

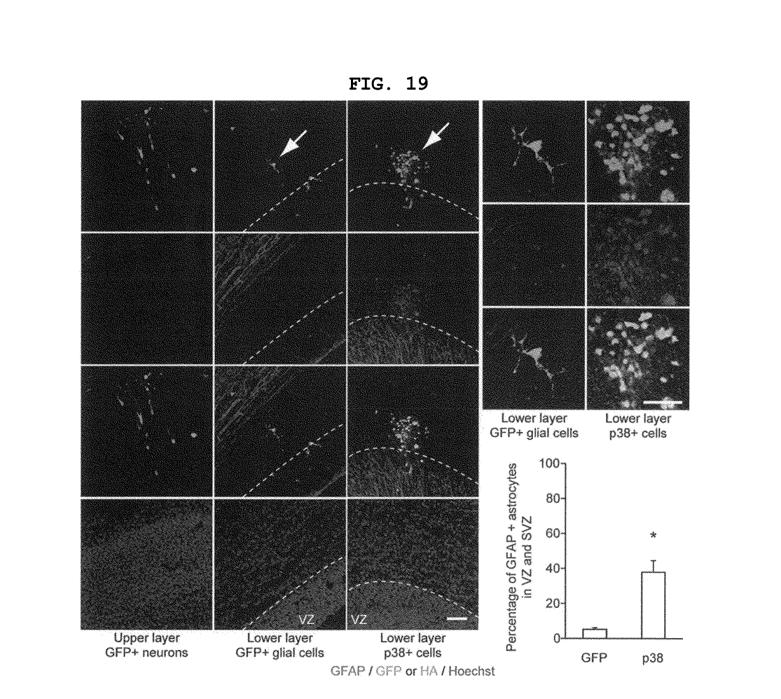

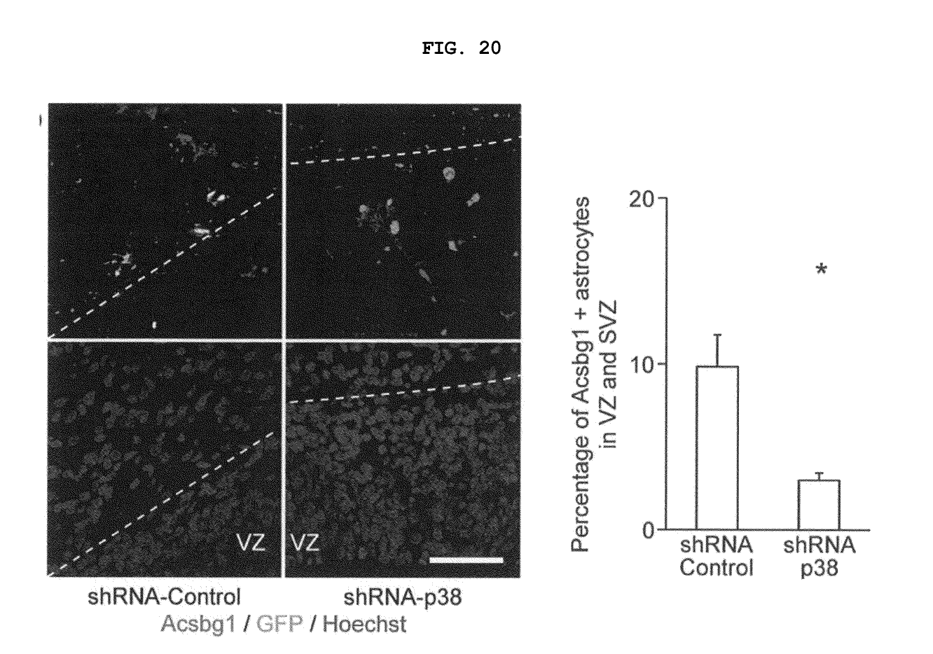

To determine the in vivo roles of p38, gain- and loss-of-function studies were performed by in utero lentiviral microinjection. Consistent with the in vitro results, when p38 was overexpressed, precocious GFAP protein expression was observed in the cerebral cortex at E17.5. In utero injections of p38-overexpressing lentivirus resulted in the generation of cell clusters with low-level GFAP protein expression in the subventricular zone (SVZ). In contrast, control cells overexpressing eGFP did not form cell clusters or express GFAP (FIG. 19). p38 knockdown experiments using a lentivirus expressing p38-specific shRNA revealed a decrease in the numbers of VZ and SVZ cells expressing acyl-CoA synthase bubblegum family 1 (Acsbg1) at E17.5 (FIG. 20). Acsbg1 is a gray matter astrocyte marker that is useful for the detection of GFAP-positive mature astrocytes and GFAP-negative immature astrocytes in the cerebral cortex (J Neurosci 28, 264-278 (2008)). These observations suggest that p38 is critically involved in the initiation of gliogenesis. Taken together, the above-mentioned results teach that the miR-17/106-p38 axis is a major regulator of the neurogenic-to-gliogenic transition in developing neural stem/progenitor cells, and that manipulation of this axis permits bidirectional control of the multipotency of neural stem/progenitor cells.

### INDUSTRIAL APPLICABILITY

According to the present invention, differentiation of neural stem cells into neurons can be promoted by new mechanism of p38 suppression. This is based on the restoration of the neurogenic competence in gliogenic neural stem cells, and neuronal differentiation can be exclusively induced.

This application is based on a patent application No. 2012-103875 filed in Japan (filing date: Apr. 27, 2012), the contents of which are incorporated in full herein.

1. An agent for promoting neuronal differentiation of neural stem cells, comprising a p38 inhibitor.

2. The agent according to claim 1, wherein the neural stem cell is gliogenic.

3. The agent according to claim 1, wherein the neural stem cell is neurogenic.

4. The agent according to claim 1, wherein the p38 inhibitor is a nucleic acid which hybridizes, under physiological conditions, to a DNA or mRNA encoding p38, thereby inhibiting the transcription and/or translation thereof, an expression vector of the nucleic acid, or a low molecular p38 inhibitor.

5. The agent according to claim 4, wherein the nucleic acid is an siRNA, miRNA or a precursor thereof, or an antisense nucleic acid targeting p38.

6. The agent according to claim 5, wherein the nucleic acid is miR-17, miR-106a, miR-106b, or a precursor of said miRNAs.

7. The agent according to claim 4, wherein the p38 inhibitor is SB203580.

8. A method of promoting neuronal differentiation of neural stem cells, comprising cultivating neural stem cells in the presence of a p38 inhibitor.

9. The method according to claim 8, wherein the neural stem cells are gliogenic.

10. The method according to claim 8, wherein the neural stem cells are neurogenic.

11. The method according to claim 8, wherein the p38 inhibitor is a nucleic acid which hybridizes, under physiological conditions, to a DNA or mRNA encoding p38, thereby inhibiting the transcription and/or translation thereof, an expression vector of the nucleic acid, or a low molecular p38 inhibitor.

12. The method according to claim 11, wherein the nucleic acid is an siRNA, miRNA or a precursor thereof, or an antisense nucleic acid targeting p38.

13. The method according to claim 12, wherein the nucleic acid is miR-17, miR-106a, miR-106b, or a precursor of said miRNAs.

14. The method according to claim 11, wherein the p38 inhibitor is SB203580.

15. A method of converting a gliogenic neural stem cell to a neurogenic neural stem cell, comprising cultivating a gliogenic neural stem cell in the presence of a p38 inhibitor.

16-20. (canceled)

21. The method according to claim 15, wherein the p38 inhibitor is a nucleic acid which hybridizes, under physiological conditions, to a DNA or mRNA encoding p38, thereby inhibiting the transcription and/or translation thereof, an expression vector of the nucleic acid, or a low molecular p38 inhibitor.

22. The method according to claim 21, wherein the nucleic acid is an siRNA, miRNA or a precursor thereof, or an antisense nucleic acid targeting p38.

23. The method according to claim 22, wherein the nucleic acid is miR-17, miR-106a, miR-106b, or a precursor of said miRNAs.

24. The method according to claim 21, wherein the p38 inhibitor is SB203580.
